(19) United States
(12) Patent Application Publication
(10) Pub. No.: US 2026/0212527 A1
(43) Pub. Date: Jul. 23, 2026

(54) IDENTIFYING CAUSAL ATTENTION OF SUBJECT BASED ON GAZE AND VISUAL CONTENT ANALYSIS

(71) Applicants: Qin Lv, Shanghai (CN); Yingying Zhao, Shanghai (CN); Yuhu Chang, Shanghai (CN); Li Shang, Shanghai (CN); The Regents of the University of Michigan, Ann Arbor, MI (US)

(72) Inventors: Qin Lv, Boulder, CO (US); Yingying Zhao, Shanghai (CN); Yuhu Chang, Shanghai (CN); Li Shang, Shanghai (CN); Robert P. Dick, Chelsea, MI (US)

(21) Appl. No.: 19/134,183

(22) PCT Filed: Dec. 1, 2022
(86) PCT No.: PCT/CN2022/135926

§ 371 Date: May 29, 2025

(51) Int. Cl. G06T 7/73; G06V 40/20

(52) U.S. Cl. CPC G06T 7/73; G06V 40/20; G06T 2207/30201

(57) ABSTRACT

A method for determining causal attention of a subject includes obtaining a first data stream indicative of one or both of i) eye movement and ii) gaze direction of the subject as the subject is viewing a scene in a field of view of the subject, obtaining a second data stream indicative of visual content in the field of view of the subject, detecting, based on the first data stream and the second data stream, an attention session of the subject, in response to detecting the attention session, determining, based on analysis of the visual content seen by the subject, a cause for behavior of the subject during the attention session, generating a causal attention output indicting the determined cause for the behavior of the subject, and causing the causal attention output to be provided to a user.

### BACKGROUND OF THE DISCLOSURE

### Field of the Disclosure

The disclosure relates generally to identifying causal attention of a subject based on gaze and visual content analysis in the field of view of the subject.

### Brief Description of Related Technology

Equipped with unmatched social cognition capabilities, domestic dogs play a variety of vital roles in modern society: pet dogs relieve stress and improve emotional welfare, guard dogs protect their companions from harm, and seeing-eye dogs assist those with impaired vision; they also protect livestock, remove pests, help their companions hunt, and find lost people. The study of dog cognition is therefore of interest to researchers working in numerous areas, and human interest in studying dogs' cognition, especially visual perceptions, has never faded. Visual perception is a widely applied cognitive clue in canine cognition research, and visual tasks are the most prevalent paradigm for examining dog behaviors. In those experiments, the experimental subjects, i.e., dogs, are required to perceive the presented visual stimulations. Then, the subject's behaviors in response to those stimuli are recorded and analyzed by human experimenters. This paradigm is commonly applied in canine vision research, including studies of object permanence, discrimination learning, spatial cognition, human-dog interactions, etc. A representative example is the classic two-choice paradigm used in Human-Dog Interactions (HDI). Two potential food sources are provided with a human demonstrator standing between them. One site contains food as a reward, while another does not. The experimenter will then instruct the dog to choose the one with the reward through certain visual human cues, e.g., pointing to the target with arms/legs, gazing at the preferable site, or turning the head. Whether dogs understand these social clues is then measured by their responses, e.g., whether they select the genuine food source.

The existing investigation methods predominantly rely on human-manipulated experiments to examine dogs' behaviors in response to visual stimuli like human body actions. As a result, those experimental paradigms are usually constrained to in-lab environments and may not reveal the dog's responses to real-world visual scenes. Besides, visual signal pertaining to the dog's responsive behavior is empirically derived from observational evidence, which can be prone to subjective bias and may lead to controversies. In the two-choice paradigm described above, for example, the entire process is manually controlled, observed, and analyzed by human experimenters. Those human-controlled experiments have considerably expanded our canine cognitive knowledge; however, overly relying on human manipulation can be a double-edged sword. For example, humans' unintentional cues can influence the behaviors and expectations of social animals, a phenomenon named the Clever Hans effect. This is especially the case in canine studies, as domestic dogs possess highly sensitive mental states and can respond to the subtle gestures made by their owners. Generally, there are two main limitations in the existing experimental protocols, e.g., the two-choice paradigm. Firstly, it is often vulnerable to the subjective bias introduced by human experimenters either consciously or subconsciously. For instance, if a human demonstrator in a two-choice HDI experiment points to the desired food site with arms or fingers as scheduled, the gaze point may fall onto another site unintentionally. This subconscious behavior can be observed and be prioritized over the pointing gesture by the dog, perhaps leading to an incorrect conclusion, although the correct conclusion would have been reached without the distraction of the demonstrator's gaze. Such unpredictable deviations can occur and accumulate without being realized by experimenters, undermining study reliability. The subjective bias can also result from the overwhelming reliance on human experts to conclude experimental outcomes, as different experimenters can judge the same phenomenon differently. Controversies and debates can emerge as a consequence, e.g., a consensus has not been achieved on the true meaning of the dog's looking-back behavior in the problem-solving task, which is interpreted as the indication of asking for help by some researchers but as a result of being attracted by the human's involuntary actions among others.

The predominantly adopted in-lab setting is another significant drawback of the current dog cognitive research. Most experimental paradigms are constrained to in-lab environments, e.g., the aforementioned two-choice protocol. Only human-controlled visual signals are delivered to dogs. This is essentially a compromise of the limited analytical scope of human experts. Dogs in the open world will indeed receive multiple complicated visual signals simultaneously and behave correspondingly. It is nearly impossible for human brains to detect and interpret which visual signal the dog is focusing on and should therefore be associated with the observed behavior. A vast majority of dog cognition studies are designed for laboratories as a result. However, experiments conducted in such a constrained environment may improperly reflect the behavioral reactions of dogs to real-world scenes pertaining to their daily duties. Although several works have described such problems and have introduced more complex scenarios involving two human instructors, the gap between laboratory and real-world conditions remains large.

### SUMMARY OF THE DISCLOSURE

In accordance with one aspect of the disclosure, a method for determining causal attention of a subject includes: obtaining, by a processor, a first data stream indicative of one or both of i) eye movement and ii) gaze direction of the subject as the subject is viewing a scene in a field of view of the subject; obtaining, by the processor, a second data stream indicative of visual content in the field of view of the subject; detecting, by the processor, based on the first data stream and the second data stream, an attention session of the subject; in response to detecting the attention session, determining, by the processor based on analysis of the visual content seen by the subject, a cause for behavior of the subject during the attention session; generating, by the processor, a causal attention output indicting the determined cause for the behavior of the subject; and causing, with the processor, the causal attention output to be provided to a user.

In accordance with another aspect of the disclosure, a method for determining causal attention of a subject includes: obtaining, by a processor, a first data stream indicative of one or both of i) eye movement and ii) gaze direction of the subject as the subject is viewing a scene in a field of view of the subject; obtaining, by the processor, a second data stream indicative of visual content in the field of view of the subject; generating, by the processor, based on the first data stream and the second data stream, a complete set comprising a plurality of graph score matrices comprising semantically meaningful information in the scene; obtaining, by the processor, a rationale set from the complete set, the rationale set comprising a subset of the graph score matrices that maximizes predictability of the behavior of the subject; determining, by the processor based on the complete set and the rationale set, causal attention of the subject viewing the scene; generating, by the processor, a causal attention output indicting the determined causal attention of the subject; and causing, with the processor, the causal attention output to be provided to a user

In accordance with yet another aspect of the disclosure, a system comprises a first sensor configured to generate data indicative of one or both of i) eye movement and ii) gaze direction of a subject as the subject is viewing a scene in a field of view of the subject; a second sensor configured to generate data indicative of visual content in the field of view of the subject; and a processor configured to: obtain a first data stream indicative of one or both of i) eye movement and ii) gaze direction of the subject as the subject is viewing a scene in a field of view of the subject, obtain a second data stream indicative of visual content in the field of view of the subject, detect based on the first data stream and the second data stream, an attention session of the subject, in response to detecting the attention session, determine, based on analysis of the visual content seen by the subject, a cause for behavior of the subject during the attention session, generate a causal attention output indicting the determined cause for the behavior of the subject, and cause the causal attention output to be provided to a user.

In connection with any one of the aforementioned aspects, the devices and/or methods described herein may alternatively or additionally include or involve any combination of one or more of the following aspects or features. Detecting the attention session includes identifying, based the first data stream, a sequence of gaze points indicating gaze direction of the subject at a plurality of time steps as the subject is viewing the scene, extracting, from the second data stream, respective score matrices for respective images in the second data stream at the plurality of time steps, and predicting, based on the sequence of gaze points and the respective score matrices, whether the subject is attentive at each time step as the subject is viewing the scene. Predicting whether the subject is attentive at a particular time step comprises computing respective differences between consecutive gaze points among the plurality of gaze points, obtaining temporal information by temporally reducing the respective score matrices corresponding to the gaze points, and generating a binary prediction indicating whether the subject is attentive based on the frame differences of gaze points and the temporal information. Determining a cause for behavior of the subject during the attention session includes generating, based on each image among a plurality of images in the second data stream corresponding to the detected attention session, a complete set comprising a plurality of graph score matrices comprising semantically meaningful information in the scene, predicting, based on the graph score matrices in the complete set, behavior of the subject viewing the scene, and determining, based on the graph score matrices in the complete set, a rationale for the predicted behavior of the subject. Each graph score matrix includes a triplet identifying a first object in the scene, a second object in the scene, and a relationship between the first object and the second object in the scene. Determining the rationale for the predicted behavior of the subject comprises generating a rationale set as subset of the complete set, the subset comprising one or more rationale score matrices that maximize predictability of the behavior of the subject, and determining the rationale by applying a trained rationale model to the rationale set. Generating the causal attention output indicting the determined cause for the behavior of the subject comprises generating a human-readable sentence that includes i) the predicted behavior of the subject and ii) the determined rationale for the predicted behavior of the subject. Generating the causal attention output indicting the determined cause for the behavior of the subject comprises converting the rationale set into a series of temporal scene graphs that illustrate changes in causal attention over time. The subject is one of i) a non-human animal subject or ii) a human subject. Determining a cause for behavior of the subject during the attention session includes determining one or more of i) associations between communicative signals detected in the scene and behavior of the subject, ii) associations between physical stimuli detected in the scene and behavior of the subject and iii) associations between content of videos and desire of the subject to watch the videos. The method further comprises, prior to generating the complete set, identifying, by the processor based on the first data stream and the second data stream, ab attention session of the subject viewing the scene, wherein detecting the attention session includes identifying, based the first data stream, a sequence of gaze points indicating gaze direction of the subject at a plurality of time steps as the subject is viewing the scene, extracting, from the second data stream, respective score matrices for respective images in the second data stream at the plurality of time steps, and predicting, based on the sequence of gaze points and the respective score matrices, whether the subject is attentive at each time step as the subject is viewing the scene. The processor is configured to detect the attention session at least by identifying, based the first data stream, a sequence of gaze points indicating gaze direction of the subject at a plurality of time steps as the subject is viewing the scene, extracting, from the second data stream, respective score matrices for respective images in the second data stream at the plurality of time steps, and predicting, based on the sequence of gaze points and the respective score matrices, whether the subject is attentive at each time step as the subject is viewing the scene. The processor is configured to determine the cause for behavior of the subject during the attention session at least by generating, based on each image among a plurality of images in the second data stream corresponding to the detected attention session, a completed set comprising a plurality of graph score matrices comprising semantically meaningful information in the scene, predicting, based on the graph score matrices in the complete set, behavior of the subject viewing the scene, generating a rationale set as subset of the complete set, the subset comprising one or more rationale score matrices that maximize predictability of the behavior of the subject, and determining, based on the graph score matrices in the complete set and the rationale set, a rationale for the predicted behavior of the subject.

### BRIEF DESCRIPTION OF THE DRAWING FIGURES

For a more complete understanding of the disclosure, reference should be made to the following detailed description and accompanying drawing figures, in which like reference numerals identify like elements in the figures.

FIG. 1 is a block diagram of an example system configured to determine causal attention accordance with an example.

FIG. 2 is a diagram depicting an example system implementation of the system of FIG. 1 using a neural network in accordance with an example.

FIGS. 3A-D depict example smart eyewear equipped with the disclosed system in accordance with an example.

FIGS. 4A-C depict, respectively. a photo of a data collection procedure, example image frames of a dog's eye image, and a scene viewed by the dog, with aligned timelines in accordance with an example.

FIG. 5 depicts time-series image frames of two scenarios in HDI application in accordance with an example.

FIG. 6 depicts time-series image frames of two scenarios in dog cognition application in accordance with an example.

FIG. 7 depicts time-series image frames of two scenarios in animal-computer interaction application in accordance with an example.

FIG. 8 is a bar chart illustration of action frequency distribution in a DogsView dataset in accordance with an example.

FIG. 9 depicts examples of two human communicative signals and the corresponding dogs' different behavioral responses.

FIG. 10 is a bar chart depiction of percentage of following human actual and misleading signals in accordance with an example.

FIG. 11 is a graph depiction of variation of mean Rationale Score (RS) for dogs' response to human's actual and misleading signals in accordance with an example.

FIG. 12 depicts two example trials of examining whether dogs follow humans' misleading communicative signals in accordance with an example.

FIG. 13 is a bar chart depiction of percentage of following a hand-point command in accordance with an example.

FIG. 14 is a graph depiction of variation of mean Rationale Score (RS) when dogs respect-response to known and new signals in accordance with an example.

FIG. 15 depicts examples of the semantic graphs when a dog follows a well-studied known action “hand pointing” and a relatively new action “leg pointing”.

FIG. 16 is a bar chart depiction of percentage of making counterproductive choice for five dogs in a food quantity performance test in accordance with an example.

FIG. 17 is a graph depiction of variation of mean Rationale Score (RS) for five dogs making counterproductive choice for five dogs in a food quantity performance test in accordance with an example.

FIG. 18 depicts examples of semantic graphs generated by the disclosed system in a trial when a dog makes counterproductive choice in accordance with an example.

FIG. 19 depicts time-series image frames for a dog when it faces a familiar person who is walking close to a door and an unfamiliar person who is running far from a door in accordance with an example.

FIG. 20 is a bar chart depicting comparison of visual attention proportion for dogs and humans in several places in accordance with examples.

FIG. 21 depicts images showing differences in visual attention in dogs and humans in several places in accordance with an example.

FIG. 22A is a picture of a dog watching projected videos in accordance with an example.

FIG. 22B is a bar chart depicting proportion of a number of frames that attract dog visual attention to a total number of frames in different video classes in accordance with an example.

FIG. 23 depicts a method for determining casual attention of a subject in accordance with an example.

FIG. 24 is a block diagram of a computing system with which aspects of the disclosure may be practiced.

The embodiments of the disclosed systems and methods may assume various forms. Specific embodiments are illustrated in the drawing and hereafter described with the understanding that the disclosure is intended to be illustrative. The disclosure is not intended to limit the invention to the specific embodiments described and illustrated herein.

### DETAILED DESCRIPTION OF THE DISCLOSURE

Systems and methods are provided that perform analysis of i) a first data stream indicative of one or both of a) eye movement and b) gaze direction of a subject as the subject is viewing a scene in a field of view of the subject and ii) a second data stream indicative of visual content in the field of view of the subject to determine causal attention of the subject on the scene. In an example, the subject may be a canine subject such as a dog. However, the disclosed systems and methods are not limited to any particular subject and may be utilized with subject other than dogs. As an example, the disclosed systems may be configured to determine causal attention of non-human subjects other than dogs viewing a scene. As another example, the disclosed systems may be configured to discover causal attention of human subjects viewing a scene.

In aspects, the first data stream may include, for example, a plurality of video frames depicting an eye gaze of the subject as the subject is viewing a scene in a field of view of the subject. The first image steam may be obtained from a first sensor, such as an inward-facing camera that may be attached to a smart eyewear frame worn by the subject or other suitable sensor. The second data stream may include one or more images or video frames capturing visual content in the field of view of the subject, for example. The second data steam may be obtained from a second sensor, such as a forward-facing camera that may be attached to the smart eyewear frame worn by the subject. In an aspect, an attention session of the subject may be detected by analyzing on gaze points and content in the vicinity of gaze points to determine whether the subject is paying attention to content in the scene. In response to detecting an attention session, content of the scene may further be analyzed. For example, as explained in more detail below, a complete set of semantically meaningful information may be generated based on the images in the second data stream representing the scene during the attentive session. The complete set may comprise a score matrix that includes a plurality of triplets identifying relationships between objects in the scene. Additionally, a rationale set may be generated as a reduced subset of the complete set. The rationale set may be the most compact subset of the complete set that can maximize the predictivity of the subject's behavior, for example.

The behavior of the subject may then be predicted based on the complete set and/or the rationale set. For example, a trained complete predictor may be used to predict behavior of the subject based on the complete set. Additionally, a trained rationale predictor may be used to predict behavior of the subject based on the rationale set. A prediction gap between the behavior of the subject predicted based on the complete set and the behavior of the subject predicted based on the rationale set may be minimized. With the minimized gap, rationale for the predicted behavior may be revealed and a causal attention output may be generated. The causal attention output may include one or both of i) a human-readable sentence and ii) a rationale graph may be generated. The human-readable sentence may be composed from the predicted behavior and the revealed rationale for the predicted behavior of the subject. As an example, in an aspect in which the subject is a dog interacting with a human, the human-readable sentence may be in the form of “The dog is performing [a predicted action] because of [the revealed causal attention].” As a more specific example, the human-readable sentence may be “The dog is staring and walking towards because a person is pointing at a paper plate.” The rationale graph, on the other hand, may comprise a graph of a plurality of relationships that may be included in the generated rationale set, for example. The rationale graph may thus depict emergence and changing of the behavior of the subject over time.

In some aspects, the disclosed systems and methods may be used to overcome the obstacles faced by decades of dog cognition research, such as subjective bias and in-lab limitations. Worn by dogs, the disclosed system captures dog visual attentive perceptions and extracts the rationale graph of visual attention, thereby providing data relevant to causal explanations of canine behavioral responses. The usability of the disclosed wearable causal attention system has been validated through in-lab evaluations. Its generality to unconstrained real-world environments has also been illustrated through in-field trials. Using the disclosed system, a large-scale dataset, sometimes referred to herein as “DogsView” dataset, containing of automatically generated causal attention estimates under a wide range of representative open-world scenarios was collected. In various examples, the DogsView dataset may be utilized to support research communities that study dog cognition, human-dog interaction, dog-computer interaction, etc., and gain insights into “the world through the eyes of dogs”.

Although the discloses systems and methods are generally describes in the context of exploring dog cognition through the lens of visual stimuli, dogs generally use multiple senses to perceive the world. The contributions of other senses, such as olfactory and auditory cues, to dog cognition are also important yet challenging for cognition investigation in dogs. Thus, the disclosed system may also be used to support the multimodal scenario to better support the exploration of dog cognition with more comprehensive capabilities, in some examples.

The disclosed systems and methods are suitable for use in wearable and/or other battery-powered and/or embedded systems, but not limited thereto. Although the disclosed systems and methods are generally described as being implemented locally in a wearable device, such as a camera system integrated with eyewear, any one or more aspects of the data processing described herein may be implemented remotely, for example at a remote server. The number, location, arrangement, configuration, and other characteristics of the processor(s) of the disclosed systems, or the processor(s) used to implement the disclosed methods, may vary accordingly.

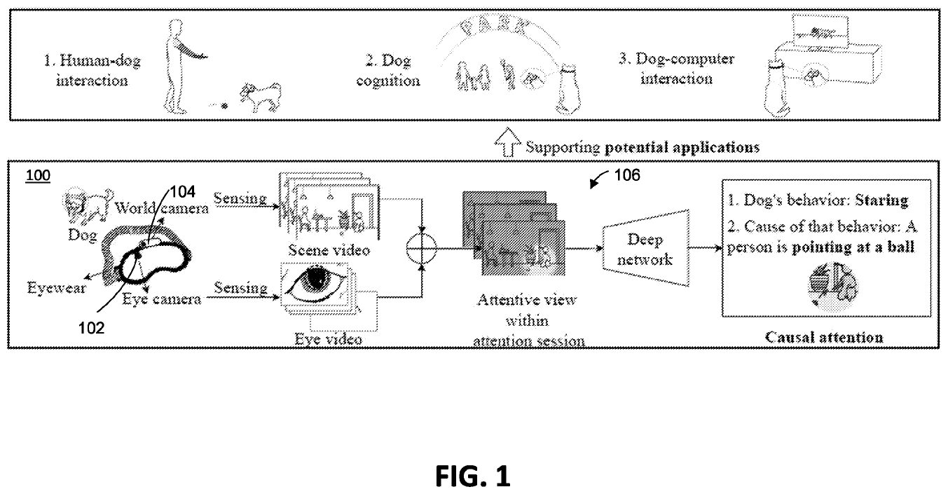

FIG. 1 is a diagram of an example system 100 configured to determine causal attention according to an example. The system 100 includes a first sensor 102 and a second sensor 104. The first sensor 102 and/or the second sensor 104 may comprises a single sensor or may comprise multiple sensors, such as multiple sensors of different types. The first sensor 102 may be a gaze movement and/or direction sensor configured to capture, detect, or otherwise obtain data indicative of eye movement and/or gaze direction of a subject (e.g., a dog) as the subject is viewing a scene. In various examples, the first sensor 102 may comprise one or more of i) a camera, such as a visible light camera, an infrared camera, etc. that may be configured to capture image or video depicting one or both eyes of the subject, ii) an infrared sensor configured to capture eye orientation based on active IR illumination of one or both eyes of the subject, iii) a camera configured to passively capture appearance of or one or both eyes of the subject, etc. Additionally or alternatively, the first sensor 102 may comprise one or more wearable position and/or orientation sensor devices, such as an accelerometer, a gyroscope, a magnetometer, etc., that may be attached to the subject (e.g., subject's head, subject's body, etc.), or to a wearable device (e.g., eyewear) that may be worn by the subject, and may be configured to detect position and/or orientation of the subject (e.g., subject's head and/or body) relative to the scene being viewed by the subject. In an example, the orientation and/or position of the subject relative to the scene being viewed by the subject may be indicative of the eye movement and/or gaze direction of the subject relative to the scene. In other examples, the first sensor 104 may additionally or alternatively comprise other suitable sensor devices that may be configured to capture or otherwise generate data indicative of one or both of a) eye movement and b) gaze direction of the subject as the subject is viewing a scene in a field of view of the subject.

The second sensor 104 may be a visual scene sensor that may be configured to capture image data, video data, etc. capturing the scene in the field of view of the subject. In various examples, the second sensor 104 may comprise one or more of i) a camera, such as a visible light camera, an infrared camera, etc., ii) a camcorder, iii) a video recorder, etc. In other examples, the second sensor 104 may additionally or alternatively comprise other suitable sensor devices that may be configured to capture or otherwise generate data, such as image or video data, indicative of visual content in the field of view of the subject.

In an example, the first sensor 102 and the second sensor 104 are mounted on eyewear, such as glasses or goggles, that may be worn by the subject, with the first sensor 102 (sometimes referred to herein as “eye camera”) configured as an inward-facing sensor, e.g., facing the eyes of the subject, and the second sensor 104 (sometimes referred to herein as “world camera”) configured as a forward-facing sensor with respect to field of view of the subject. In other examples, instead of being attached to a subject or to a device worn by the subject, the first sensor 102 and/or the second sensor 104 may be located at a suitable distance from the subject. For example, the first sensor 102 and/or the second sensor 104 may be a distance sensor (e.g., distance camera) positioned in the vicinity of the subject. As just an example, the first sensor 102 may be a web camera, or webcam, that may generally be facing the subject as the subject is viewing the scene.

In an example, the second sensor 104 is configured to capture images (e.g., video) or other data indicative of a scene being viewed by the subject, whereas the first sensor 102 is configured to concurrently capture images or other data indicative of the subject's gaze as the subject is viewing the scene. The system 100 may be configured to analyze the scene images (or other data indicative of visual content in the scene) captured by the second sensor 104 and the eye images (or other data indicative of subject's gaze) captured by the first sensor 102 to identify causal attention of the subject. In examples, determining the causal attention of the subject is defined as the process of identifying visual signal(s) that can lead to particular behavioral responses of the subject. In an example, the system 100 may include a causal attention engine 106 configured to analyze the scene images and the eye images to identify attention sessions and of the subject, and identify attentive view within the attentive sessions in the field of view of the subject. The causal attention engine 106 may be implemented on or by a processor, such as one or more of a central processing unit (CPU), a graphical processing unit (GPU), a microcontroller, digital signal processor (DSP), a machine learning accelerator, etc. The processor implementing the causal attention engine 106 may be any suitable processor that may be implemented on one or more integrated circuits, such as application specific integrated circuit (ASIC), field programmable gate arrays (FPGA), etc. The causal attention engine 106 may include one or more models, such as one or more deep neural networks, trained or otherwise configured to determine a causal attention engine 106 that may be configured to detect such as a deep neural network, trained or otherwise configured to analyze the attentive view with the attentive sessions in the field of view of the subject, and predict i) the behavior of the subject and ii) the cause of the predicted behavior of the subject. Generally, previous works in dog cognition are the applications of eye-tracking techniques to analyze the gazing behavior of dogs. The obtained gaze points are seen as indicators of canine visual attention and the gaze patterns are used to analyze the dog's visual cognition of human faces, objects within pictures, the implications of oxytocin, and so on. Simple as it is from a technology perspective and even without deep networks, the works of this branch have already yielded several interesting discoveries. For example, it has been discovered that canine visual attention generally focuses on more informative regions in pictures, while facial images of conspecifics are preferred over other objects. Despite these findings, previous canine eye-tracking techniques can only detect the gaze point, which can only provide a rough estimate of the dog's observation. Thus, in current systems, the presented visual stimuli still need to be carefully selected by humans to avoid potential ambiguities, making these techniques not suitable for comprehending real-world canine visual attention in detail.

In examples, the system 100 is configured to analyze the causal attention in dog visions, e.g., by accurately determining whether the dog is in a visually attentive state (looking at something attentively) and discovering the visual perceptions that lead to its behavioral response. That is, the system 100 may identify the visual attention that can reveal the causes of a dog's behavioral responses. Awareness of causal attention allows canine cognitive experiments to be conducted in unconstrained environments where dogs can behave more naturalistically and realistically. It can also provide an objective evaluation of the specific visual signals that produce particular behavioral responses and, therefore, can significantly eliminate those potential subjective biases in dog cognitive studies.

Revealing such causal attention, however, is not a straightforward task. Considering, for example, a simple two-choice experiment, i.e., a person points to a ball, and then the dog walks towards the ball as expected, the former can be deemed as the cause of the latter. In an example, the system 100 is configured to explicitly determine what is observed in the dog's eyes, especially in terms of semantic meanings, e.g., (person-pointing to-ball) in an example. In an aspect, the system 100 is configured to express what is observed in the dog's eyes as a simple triplet graph, allowing the system 100 to discover causal relationships between what is observed in the dog's eyes and the dog's demonstrated behavior. In real-world scenarios, there can be multiple such triplet graphs, each corresponding to a different visual signal perceived by the dog and serving as a possible cause. Determining causal attention signal(s) can be challenging; dogs cannot verbally describe the reasons for their behaviors. The behaviors may be discovered following visual attention.

In examples, the system 100 is configured to interpret the visual attention of dogs, e.g., human body actions under HDI scenarios, as a semantic graph consisting of multiple triplets. Each triplet is in the form of (subject-relationship-object) and stands for a perceived visual signal, for example. As described in more detail below, relying on an effective assumption to estimate causal attention, the system 100 may aim to find the minimum set of the visual attention graph that is most informative to predict the dog's behavioral responses. The resulting set is sometimes referred to herein as a “rationale graph”. The rationale graph is the most compact graph that maximizes the probability of predicting the dog's responsive behaviors.

The system 100 may, for example, include a rationale graph generator that is integrated with the causal attention engine 106 and is configured to obtain the rationale graph from a dog's visual attention. The training of the causal attention engine 106, as a result, has a specific requirement for data annotations, e.g., whether the dog is watching attentively, the type of dog behaviors following an attentive session, and the ground-truth causality, in various examples. In an example, a dataset is collected under the HDI scenario to satisfy the training requirement of the causal attention engine 106, and all the required information is manually annotated in the collected dataset. The annotated dataset is used to train the causal attention engine 106, in an example. The performance of the disclosed system surpasses that of other baseline methods. To facilitate dog cognition research, the disclosed system has been applied to a wide range of representative experimental scenarios, including the two-choice experiment, the dog's understanding of misleading signals, and a food quantity preference test. Dogs' visual attention and causal attention in those sessions are automatically inferred by the disclosed system. In an aspect, a dataset (sometimes referred to herein as “DogsView” dataset) that comprises the inferences is generated. The DogsView dataset may to facilitate relevant research on dog cognition, HDI, dog-computer interaction, and more. In aspects, the rationale graph predicted by disclosed system is consistent with the causality estimates of expert human analysts, justifying that causal attention may be identified by the generated rationale graph. In-field trials have been performed to investigate the performance of the disclosed system under additional real-world scenarios, demonstrating its applicability over unconstrained environments.

In examples, the disclosed systems and methods address the core issue of visual causal attention for dogs. Generally, the patterns of a dog's eye movements are similar to those of a human, which both can be divided into distinct stages of saccades, smooth pursuits, and fixations. However, the eye movements of humans and dogs are not identical. A notable difference is that the movements of dogs' eyes, when compared with those of humans, typically exhibit longer fixation periods and shorter saccade durations. In examples, this timing difference is considered when applying human eye-tracking methods to dogs to reflect the biological deviations better.

The disclosed systems and methods may rely on associations between the patterns of a dog's eye movement and its attentive states. For example, the disclosed systems and methods may examine whether a dog can focus on the informative region of an image via examining its gazing behaviors. In examples, if fixations of eyes are detected, the dog is considered to be attending to the gazing region. On the other hand, smooth pursuit is also a significant indicator of a dog's attention. In some examples, the disclosed methods and systems thus consider both fixation and smooth pursuit as the proxy for visual attention of dogs, which is generally similar to human attention. In various examples, canine visual attention is defined as a function of three conditions: (1) the dog's current eye movement pattern is either smooth pursuits or fixation, (2) the duration of the current gaze pattern has exceeded a threshold, and (3) there is a meaningful visual stimulus near the gaze location. The motivations of the first two requirements are to ensure dogs are gazing attentively. The last criterion excludes cases where dogs are staring at nothing, as it is a clear sign of being mentally unfocused; It also allows the system to focus on the central visual field of the dog's vision, which may be captured at higher resolution than the peripheral visual field. With all three conditions satisfied, it may be concluded that the dog is attentively looking at something meaningful, i.e., visual attention is established. The system may then proceed to analyze the attended visual stimuli, in an example.

The visual systems of dogs and humans are not identical. Physiologic studies have clarified that dogs' eyes are different from the eyes of humans in several ways, including color perception, sensitivity to light, and visual acuity. In aspects, the disclosed system is configured to discover the implications of visual stimuli and attention processing for dog cognition and behavior studies. In aspects of the present disclosure, a visual stimulus is defined to be a particular vision, either short-term or long-term, that may lead to a behavioral response. Typical examples include, but are not limited to, the body actions of the human companion, bouncing balls, flying insects, etc. Such signals may be categorized into social or non-social cognition, depending on whether a human is involved. In examples described herein, human body motion in HDI scenarios is generally used as the visual stimuli, which fall into the social cognition domain, merely for illustrative purposes. Other visual stimuli, whether social or non-social, can be addressed in other aspects.

Referring still to FIG. 1, in an example, the causal attention engine 106 is trained or otherwise configured to determine causal attention of a non-human animal subject, such as a canine subject (e.g., a dog) or other non-human subject, in various scenarios based on gaze and visual content information obtained from the first sensor 102 and the second sensor 104. In some examples in which the subject is a canine subject, the causal attention engine 106 may be trained or otherwise configured to determine a cause for behavior or other response of a canine subject by determining one or more of i) associations between human communicative signals detected in the scene and behavior of the canine subject, ii) associations between physical stimuli detected in the scene and behavior of the canine subject and iii) associations between content of videos and desire of the canine subject to watch the videos. In other examples, the causal attention engine 106 is trained or otherwise configured to determine causal attention of a human subject. For example, the causal attention engine 106 may be trained or otherwise configured to determine a cause for behavior or other response of a human subject to what is happening in the scene, such as actions performed by other humans or non-human animals in the scene or other signals that may be perceived by the human subject viewing the scene. In some examples, the causal attention engine 106 may be trained or otherwise configured to determine causal attention, such as reasons for behavior or other responses, of a human or non-human animal subject in various “multi-actor” scenarios in which the subject may be responding to one or more other actors in the scene.

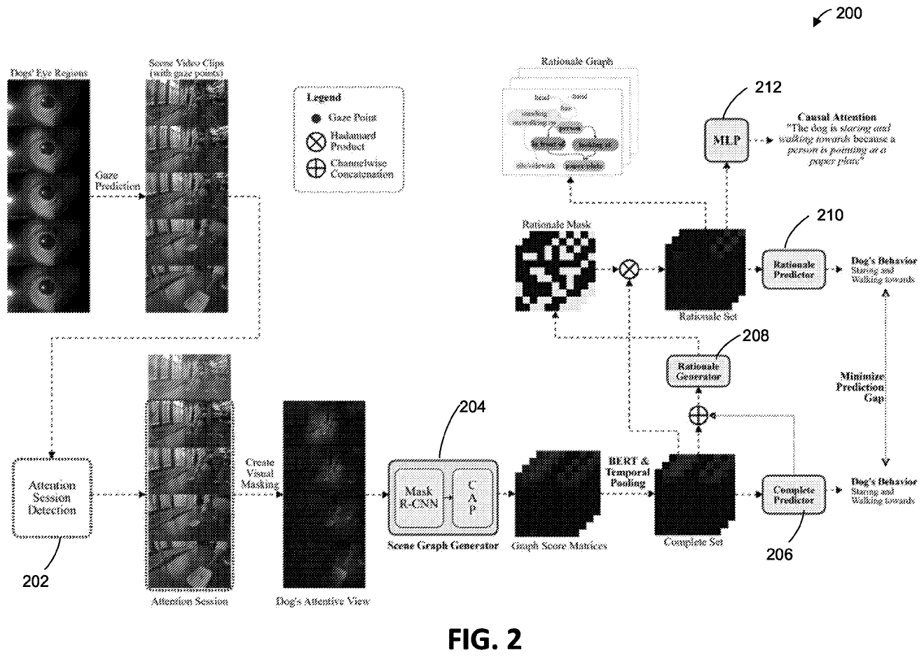

Turning now to FIG. 2, a diagram depicting an example implementation of a system 200 configured to determine causal attention is provided in accordance with one example. The system 200 generally corresponds to, or is utilized with, the system 100 of FIG. 1 in some examples. For example, the system 200 corresponds to, or is included in, the causal analysis engine 106 of FIG. 1. In an example, inputs to the system 200 include i) dog's eye regions captured by the inward-facing eye camera and ii) scene images captured by the outward-facing world camera. The system 200 may include an attention session detector 202 that may be configured to analyze gaze time-series and scene images to determine the durations in which the dog is visually attending to something within the scene. For each detected visual attention session, the system 200 may apply visual masking to each scene image to highlight the regions near the gaze points and to reduce the significance of less relevant parts. The masked scene images may be fed into a Scene Graph Generator (SGG) 204 to extract a score matrix (a matrix containing posterior probabilities of all semantic triplets) of scene graphs. A BERT model and a temporal pooling method may then be used to refine the score matrix and to aggregate the temporal information to a fixed value. In an example, the resulting score matrix contains all the possible scene semantics that can lead to the dog's responsive behaviors, and therefore the resulting score matrix may be defined as the “complete set.” A complete predictor 206 may then be utilized to predict the dog's behavior from the complete set.

The system 200 may include a rationale generator 208. The rationale generator 208 may be trained or otherwise configured to reveal the rationales from the complete set. In an example, the output of the rationale generator 208 is a binary mask (or rationale mask) of the same size as the complete set. The mask may be multiplied element-wise with the complete set to obtain a masked set, sometimes referred to herein as the “rationale set,” or the rationale score matrix in particular. In various examples, the rationale set is intended to be the minimum set that can maximize the predictivity of the dog's behavior. The system 200 may thus include a rationale predictor 210 that may be trained or otherwise configured to predict the dog's behavior based on the rationale score matrix. The system 200 may be configured to minimize the prediction gap between the complete predictor 206 and the rationale predictor 210.

In an example, when produced by a well-trained rationale predictor 210, the rationale set is the most compact set that consists of the maximum amount of semantic information regarding the dog's responsive behavior, and it can be visualized in the form of scene graphs, or the rationale graph. In an example, the system 200 may also include a causal attention output generator 212 which may comprise, for example, a Multi-Layer Perceptron (MLP) model, configured to summarize the rationale score matrix into a single human action triplet (e.g., a human action triplet in a Human-Dog Interactions scenario) to express causal attention in a short, human-readable sentence.

In an example, the attention session detection engine 202 is configured to find the temporal sessions during which the dog is visually attentive based on analyzing the dog's gaze patterns and the scene images. At a time step t, video frames in time range [t−N, t+N] may be considered to be the most relevant frames and information from this range may be used to predict the attentive state in step t. Let E^{t}={E_{t-N}, E_{t-N+1}, . . . , E_{t+N}} be the dog's eye image sequence of this range where Eᵢ refers to the eye image at step i, and let I^{t}={I_{t-N}, I_{t-N+1}, . . . , I_{t+N}} denote the scene sequence of the same range where Iᵢ is the scene image at step i. In an example, the attention session detection engine 202 is configured to obtain the gaze position from each eye image. The attention session detection engine 202 may be configured to obtain, from the eye sequence Eₜ, a sequence of gaze points g^{t}={g_{t-N}, g_{t-N+1}, . . . , g_{t+N}}, where gᵢ is gaze position at time step i. The scene sequence I^{t} is provided to the Scene Graph Generator (SGG) to extracts a score matrix from each image, i.e., sᵢ=N_{SGG}(Iᵢ) where sᵢ represents the score matrix at step i and N_{SGG} denotes the SGG network, and denote the score matrices of the sequence as s^{t}={s_{t-N}, s_{t-N+1}, . . . , s_{t+N}}.

With gaze points g^{t} and score matrices s^{t} obtained, the system 200 may predict whether the dog is visually attentive at time step t. In an example, the system 200 may utilize Temporal Pooling (TP) to score matrices s^{t} to summarize the temporal information in the scene sequence. Also, the system 200 may compute differences between consecutive gaze points to summarize the gaze movement. The temporally reduced score matrix and frame differences of gaze points may be concatenated together and then fed into a Multi-Layer Perceptron (MLP) to produce a binary prediction, i.e., whether the dog is visually attentive in time step t. In an example, the binary prediction process is performed in a frame-wise manner, and, for each frame, the system 200 may predict whether the dog is visually attentive. The system 200 may determine that a temporal session is an attention session if the dog is visually attentive in most of the frames, for example.

In examples, detection of an attentive session may trigger analysis of causal attention. After an attention session is detected, the system 200 may attempt to find the dog's behavioral causality in this session. To this end, the system 200 may utilize a complete predictor to predict the dog's behavior from all possible semantics of the dog's visual attention. In an example, if there are a total of K attention sessions during training and I^{j}={I₀, I₁, . . . , I_{T}} is the scene sequence of the j-th attention session of duration T+1 where j∈[0, K)∩Z, and g^{j}={g₀, g₁, . . . , g_{T}} is the predicted gaze points of the same session, Gaussian Relaxation may be applied to convert those gaze points g/into heatmaps and the heat maps may be applied as visual masks on the corresponding scene images. The system 200 may thus focus the model on regions closer to the gaze points, as the locations near the gaze points are areas of potential interest to subjects. This can be denoted as I′=VM(li, gi) where I′ is the masked scene at step i, and VM refers to this Visual Masking processing. The masked scene sequence can be denoted as Ij′={I′, I′, . . . , l′}.

After visual masking, the system 200 may employ a Scene Graph Generator (SGG) to extract the score matrix of the scene graph from each scene image. In an example, the SGG comprises a Faster R-CNN and a Casual Analysis Predictor (CAP). If the SGG network is denoted as N_{SGG}, then sᵢ=N_{SGG}(I′) where sᵢ is the score matrix at step i. To filter semantically meaningless triplets in the extracted graph score matrices, a BERT model may be applied to s^{j}, i.e., s^{j}′=N_{BERT}(s^{j}), where s^{j}={s₀, s₁, . . . , s_{T}} are the extracted score matrices for this attention session and s^{j′} is the filtered score matrix and N_{BERT} is the BERT model. Since attention sessions may have different lengths, Temporal Pooling (TP) may be applied to s^{j′} to reduce the time dimension to be a fixed value (e.g., 10), i.e., C^{j}=TP(s^{j′}), where C^{j} is the temporally reduced graph score matrix and TP is the Temporal Pooling operation.

In an example, the extracted score matrix C^{j} contains all semantically meaningful information that is related to the dog's behavior. Thus, the extracted score matrix C^{j} is sometimes referred to herein as the “complete set”. The extracted score matrix C^{j} may be provided a complete predictor 208 configured to predict the dog's behavior from the complete set. In an example, the complete predictor 208 may comprise an MLP model consisting of a linear layer and a soft-max layer. In other examples, other suitable models may be utilized. If Nc denotes the complete predictor, then y\hat{ }^{j}=N_{C}(C^{j}), where y\hat{ }^{j} is the predicted type of canine behavior based on the complete set. Further, if y^{j} is the ground-truth of the dog's behavioral response for this session. The complete, a Cross-Entropy loss may be defined to train the complete predictor, which can be written as:

$${ℒ}_{C} = {\sum }_{j} CE ( {y}^{j} , {\hat{y}}_{C}^{j} ) , \qquad Equation 1$$

where L_{C} is the loss of the complete predictor over all attention sessions during training, and CE is the Cross-Entropy loss.

Because, in an example, the complete set contains all the semantic information, the complete set may be heavily redundant, making it difficult to retrieve a reasonable behavioral causality, in at least some cases. A rationale generator and predictor may thus be provided to reveal the pursued causal attention. As mentioned above, a rationale set may be a learned most compact subset of the complete set, that can maximize the predictivity of the dog's behavior. A rationale generator may first be applied to the complete set C^{j} to generate a binary mask from the complete set C^{j} and also from the weights of the linear layer of the complete predictor N_{C}. The binary mask may have the same size as the complete set and indicates whether each element in the complete set should be kept or discarded. The rationale generator, N_{G}, may be an MLP model or other suitable model. In an example, the weights of the linear layer in the complete predictor N_{C}, W_{C}, may be concatenated with C^{j} and then provided to the rationale generator N_{G} to obtain a rationale score, i.e., r^{j}=N_{G} ((C^{j}⊕W_{C})), where ⊕ refers to channel-wise concatenation and r^{j} refers to the predicted rationale score. The rationale score matrix r^{j} may be the same size as the complete set C^{j}, and each element of r^{j} may have a normalized value between [0, 1).

Generally, a larger rationale score value in r^{j} indicates that the element of the same position in C^{j} may have a higher probability of being the rationale, i.e., being more relevant to causal attention. In an example, r^{j} may be rounded to a binary rationale mask b), and then multiplied, element-wise, with the complete set C^{j} to produce the rationale set, i.e., R^{j}=b^{j}⊗C^{j}, where R^{j} stands for the rationale set, ⊗ is the Hadamard product. In an example, the rationale set R^{j} may be provided to a rationale predictor, N_{R}, configured to predict the dog's behavioral response. The rationale predictor NR may be is an MLP model or other suitable model. The predicted dog behavior based on the rationale set may be denoted as y\hat{ }^{j}=N_{R}(R^{j}). Similar to the complete predictor's loss, a Cross-Entropy loss for the rationale predictor may be defined as:

$${ℒ}_{R} = {\sum }_{j} CE ( {y}^{j} , {\hat{y}}_{R}^{j} ) , \qquad Equation 2$$

where L_{R} is the loss of the rationale predictor over all training sessions. However, L_{R} is not the only loss that is used to train the rationale generator N_{G} and the rationale predictor N_{R}, in at least some examples. In an example, there are two major components to training the system 200: the complete predictor N_{C}, and the rationale-related networks, i.e., the generator N_{G} and the predictor N_{R}. Because training the complete predictor N_{C} does not reply on N_{G} or N_{R}, the loss L_{C} in Equation 1 may first be invoked to train N_{C}, temporarily ignoring N_{G} and N_{R}. After the complete predictor N_{C} is trained, the weights of the complete predictor N_{C} may be frozen for simplicity and training of the rationale generator and predictor may be continued. Training may include minimizing the prediction gap between the complete predictor and rationale predictor since the rationale set should maintain the maximum predictivity from the complete set and the predictivity of the rationale set should be as close to that of the complete set as possible. This can be written as

$${ℒ}_{R , C} = ReLU ( {ℒ}_{C} - {ℒ}_{R} ) , \qquad Equation 3$$

where ReLU is the ReLU operation. In aspects, Equation 3 generally ensures that the predictivity of the rationale set is as close to that of the complete set as possible.

In an example, the rationale set is determined such that the rationale set is a most compact set of the complete set. To this end, another regularization loss may be added:

$${ℒ}_{Reg} = 𝔼 ( {‖ R ‖}_{1} ) - η , \qquad Equation 4$$

where η is a pre-defined gap level, and E(∥(R)∥₁) is the proportion of the non-zero elements in all rationale sets. In other words, a rationale set ghat is as sparse/compact as possible is determined.

The final loss used to train the rationale generator and rationale predictor can thus be written as:

$$\begin{matrix}{L}_{G} = {ℒ}_{R} + {αℒ}_{R , C} + {βℒ}_{Reg} , \\ = {\sum }_{j} CE ( {y}^{j} , {\hat{y}}_{R}^{j} ) + α ReLU ( {ℒ}_{C} - {ℒ}_{R} ) + β ( 𝔼 ( {‖ R ‖}_{1} ) - η ) ,\end{matrix} \qquad Equation 5$$

where LG represents the final loss to train NG and NR, α, and β are the weights of different loss terms, respectively.

The trained system 200 network may generate the rationale set Rj for an attention session. Further, an MLP network may be applied to Rj to capture the causal attention that can be intuitively understood by humans. The MPL network may be configured to output the type of visual stimulus that can be seen as the cause of the dog's behavioral response. As an example, in HDI scenarios, the MPL network may output different types of human actions that can be seen as the cause of the dog's behavioral response. In other examples, other dog cognition scenarios may be utilized and corresponding other types and the MPL network may generate other types of outputs that capture the dog's causal attention. The causal attention of dogs may thus be described in clear, human-readable sentences. Because the expected behavior of the dog has already been provided by the complete/rationale predictor, the expected dog behavior may be assembled with the behavioral causality and predict the causal attention as, e.g., “The dog is staring and walking towards because a person is pointing at a paper plate.”

The rationale set Rj also reveals how the predicted causal attention occurs temporally, in at least some examples. For example, the rationale set Rj may be converted into a series of temporally consistent scene graphs to intuitively explain how the causal attention emerges and changes.

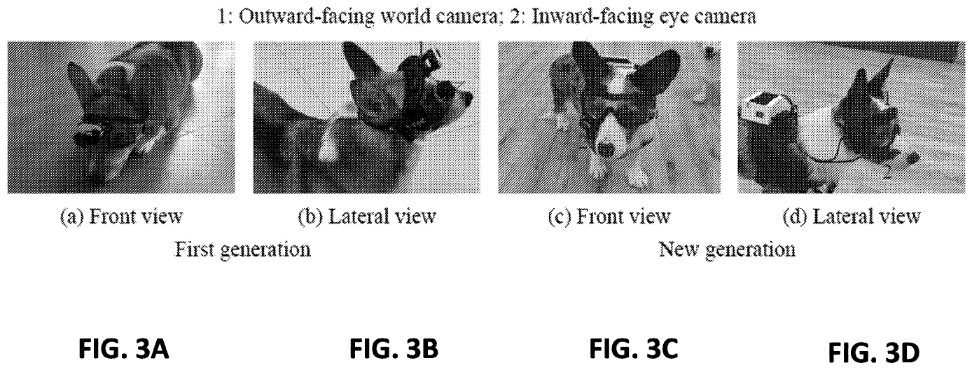

Turning now to FIGS. 3A-D, example smart eyewear (e.g., smart glasses or smart goggles) equipped with the disclosed system, in accordance with examples, are depicted. In particular, FIGS. 3A-B depict, respectively, a front view and a lateral view, of a dog wearing an example implementation of smart glasses equipped with the disclosed system according to one example. Similarly, FIGS. 3C-D depict, respectively, a front view and a lateral view, of a dog wearing another example implementation of smart glasses equipped with the disclosed system according to another example. In examples, the smart glasses are designed to be lightweight for the sake of comfort. The example smart eyewear may be designed to suit the use of canines to ensure that wearing eyewear will not significantly change the behaviors of dogs. To this end, various strategies may be employed, including, for example, 1) developing a lightweight and comfortable hardware prototype and 2) using a carefully designed training paradigm that allows dogs to acclimate to the eyewear. In an aspect, example smart eyewear 400 is retrofitted from commercially available goggles specially designed for dogs. For example, two lightweight cameras-one inward-facing eye camera and one outward-facing world camera—may be attached.

In an example, SHETU SQ11 1080p camera module (1920×1080@30 fps) may be adopted as the eye camera. In some cases, an additional IR LED light may be utilized to light up the iris regions. The world camera may be an Insta360 GO 2 (1920×1080@30 fps) camera that collects the visual contents adjusted with the canine's field of view (FoV). In an aspect, the cameras may capture a suitable number of frames per second (e.g., 30 frames per second) that us adequate for detecting dig eye movement patterns such as fixations, the durations of which are typically longer than 100 milliseconds (ms). A higher frame rate would unnecessarily reduce battery lifespan, in at least some examples. The video streams from the two cameras may be synchronized by aligning their time stamps.

As compared to the example implementation depicted in FIGS. 3A-B, the mechanical design in the implementation in depicted in FIGS. 3C-D is overhauled to reduce weight, optimize mechanical reliability, and improve comfort. A key modification is detaching the battery and the control board from the headset and organizing them into a small backpack carried by the dog. This modification reduces the weight of the headset to improve comfort. In an example, the resulting headset weighs approximately 72.4 g, and the weight of the backpack is 369.8 g.

In some cases, the discloses system may generally operate offline. For example, the headset may record the video streams with the two cameras while the smart eyewear is worn by the dog. In an aspect, the recorded data may be uploaded to a cloud infrastructure that may be configured to perform more computation-intensive tasks such as model inferences.

In an aspect, the disclosed system is utilized to construct a dog egocentric vision dataset, sometimes referred to herein DogsView dataset, to support research communities studying dog cognition, HDI, dog-computer interaction, and more. For example, data may be collected using the disclosed systems and methods over a certain period of time, such as several months, and in various environments, such as open-world scenarios. The data mat then be annotated, for example to add HDI signal annotations. The DogsView dataset may cover a wide range of applications and may be designed to overcome key obstacles faced by current research on dog cognition, HDI, and dog-computer interaction, such as (1) the potential subjective bias of manual analysis results and (2) the limitations of in-lab experiments. Understanding the visual cognitive abilities of dogs is a challenging problem because its scope is broad. The disclosed systems and methods automatically capture visual attention in dogs and discover the rationale for visual stimuli that lead to their behavioral responses in the open world. The disclosed systems and methods and the DogsView dataset may enable interdisciplinary HDI research.

The DogsView dataset may target representative applications regarding dog cognition, HDI, and dog-computer interaction. Dog egocentric scene videos and eye-tracking videos may be recorded with aligned timelines for representative applications. The egocentric scene videos may cover dog visual scenes, which may be used for visual semantic understanding by extracting objects (e.g., human subjects) and interactions in dog visual scenes using image and video analysis. Eye-tracking videos may capture dog visual attention. Scene and eye-tracking videos may be aligned spatially and temporally to reveal dog visual attention over time, identify the corresponding HDI signals, quantify the rationale score of each signal, and then support canine visual causal reasoning.

The DogsView dataset may be automatically annotated in terms of frame-by-frame visual attention of dogs, human communication signals (i.e., body actions) and representative scene graphs for each attention session. For example, a number (e.g., twenty or more) types of human communicative signals in the context of human-dog interactions may be utilized. Such signals may contain number human communicative signals (e.g., seven typical signals) such as hand gestures and eye-gaze contact. Additional new signals (e.g., fourteen new signals) may be added that have been previously ignored due to the limitations of existing studies. This rich set of features may enable researchers to gain insight into dogs' visual cognition by comprehensively and quantitatively measuring how human communicative signals affect dog behaviors, and further reveal why and how dogs continually learn from unseen signals.

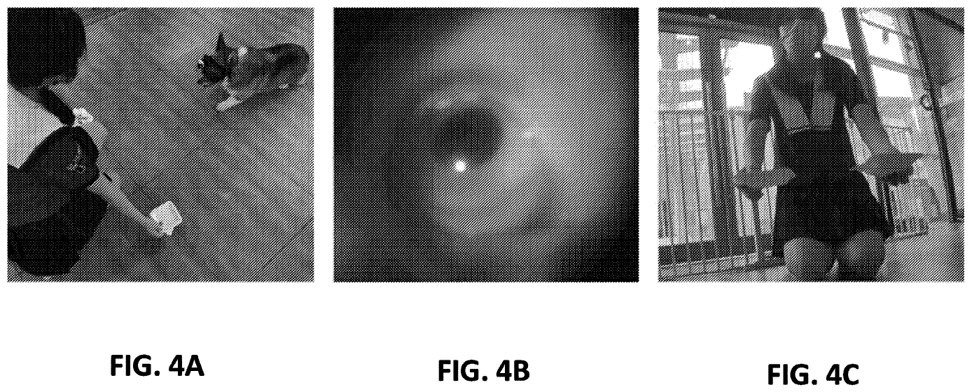

A training paradigm used for collection of that data may include acclimating a dog to wearing glasses and maintaining the dog's mental status in appropriate conditions. In each data collection session, an experimenter may bring the dog to the target location. At the beginning of the session, the dog may be allowed to act freely, typically barking and running, for a certain among of time (e.g., around 20 minutes) to adapt to the new environment. The smart eyewear system may then be placed on the dog and the dog may be rewarded for wearing the smart eyewear with treats and human praise. The dog may be allowed to acclimate to the device to reduce disturbance from wearing the hardware. After the dog behaves naturally for a certain period of time (e.g., a few minutes) without any stress signals, the disclosed system may be turned on to begin data collection. Human experimenters may observe the behaviors of the dog to ensure that the dog is in an appropriate mental state during the data recording. If any unusual signals are spotted, such as the dog lying on the ground even after it is rewarded, the recording may be stopped and corrective actions (e.g., replacing the tired dog with the one full of energy) may be taken. Turning briefly to FIGS. 4A-C example data collection according to an example is depicted. In particular, FIG. 4A depicts a dog interacting with a human participant, and FIGS. 4B-C depict an example of the captured eye images and scene images with aligned timelines, respectively.

In aspects, the DogsView dataset covers at least the following three representative applications related to research on dog cognition, human-dog interaction, and dog-computer interaction. Human communicative signals that are easily perceived by dogs and trigger their interactive behavioral responses have long been studied. However, the types of signals identified and extensively studied are limited, mostly focusing on hand pointing, eye-gaze contact (or eye glances), back-turning, and arm extension. In addition, prior research has studied whether dogs can recognize misleading communicative signals from humans and continuously learn new human communicative signals. For example, a previous study found that dogs do not blindly follow misleading human behaviors, such as pointing gesture.

In aspects, a human-dog interaction application may be designed with at least two goals: (1) exploring the visual cognitive processes in dogs perceiving, encoding, and processing misleading communicative signals from humans, and further inferring why dogs can recognize these signals and (2) automatically and quantitatively explaining how dogs learn from new/unknown human communicative signals, and inferring possible causes. In an example, the human-dog interaction application is designed as a two-step data collection process. The first step involves manually designed human communicative signals, including actual, misleading, and new/unseen human communicative signals for dogs. In contrast, the second step is exploratory. For example, dogs are placed into unconstrained open-world scenarios to collect data on their visual attention, aiming to explore and discover potentially interesting but unknown human interaction cues.

The human-dog interaction generally focuses on the social aspect of dog cognition. In other aspects, non-social aspects of dog cognition related to visual stimuli may be investigated. For example, a non-social dog cognition application may explore how dogs perceive the physical stimuli of their surroundings, resulting in decisions producing behavioral responses. The importance of non-social aspects of dog cognition has been demonstrated in the past in laboratory settings, but the scope of the previous work is limited in physical stimuli and environmental diversity. In a non-social dog cognition application, the experimenter may be instructed to take the dog outdoors to experience various daily life environmental scenarios. During this process, the experimenter and the dog may interact with the environment freely. During the process, the disclosed system may be used to collect the visual attentive content of dogs in the open world, and automatically analyze dog visual attention as well as the reasons behind it.

In another example, a human-computer interaction application may be explored. An important subfield of human-computer interaction (HCI), research into animal-computer interaction has investigated how dogs interact with video content, which can potentially cover a wide range of real-world social and non-social scenarios. In an example human-computer interaction application, a dog may be taken into a room with an LED screen playing short videos collected from an online platform, such as, for example, Tik Tok or YouTube. A person familiar with the dog may stay in the same room to make sure the dog is comfortable and relaxed. The dog may be free to decide whether to watch the video or not. The disclosed system may be used to collect and record visual attention data for dogs watching video segments. The videos may be classified into a number (e.g., four) categories based on their content. In an example, one or more categories (e.g., three categories, in an example) are socially relevant, such as humans, dogs, and other animals. Other one or more categories (e.g., one category, in an example) are non-socially relevant, containing inanimate objects. To enable a fair comparison, an equal number of segments, with similar durations, from each video category, may be played. As just an example, six video segments and four minutes per category on average may be played. In some cases, to ensure a diversity of visual stimuli, each category may consist of multiple (e.g., three or more scenarios). For example, in the animal category, four kinds of animals, e.g., tigers, wolves, and ducks mat be shown.

Table 1 summarizes the DogsView dataset, according to an example. In the illustrated example, the DogsView dataset includes a total of 213.00-minute timeline-aligned eye-scene videos are constructed, including 63 pairs of eye-scene video clips. The scene video has a spatial resolution of 1,920×1088 pixels, while the eye video has a spatial resolution of 1,920×1080 pixels. Both videos have a sampling rate of 30 fps.

TABLE 1

| | | | | # of human | Hassan | | Dog |
|---|---|---|---|---|---|---|---|
| | Representative | Video | # of | action | actions | Attention | behavior |
| Applications | scenarios | minutes | videos | categories | annotated | annotated | annotated |
| Human-Dog | Various | 124.30 | 38 | 21 | ✓ | ✓ | ✓ |
| Interaction | communicative | | | | | | |
| (HDI) | signals from | | | | | | |
| | humans for dogs | | | | | | |
| Dog | In pet stores; | 68.27 | 11 | 21 | ✓ | ✓ | ✓ |
| Cognition | shopping malls, | | | | | | |
| | and parks | | | | | | |
| Animal- | Socially relevant | 20.43 | 14 | 13 | ✓ | ✓ | ✓ |
| Computer | and non-socially | | | | | | |
| Interaction | relevant | | | | | | |

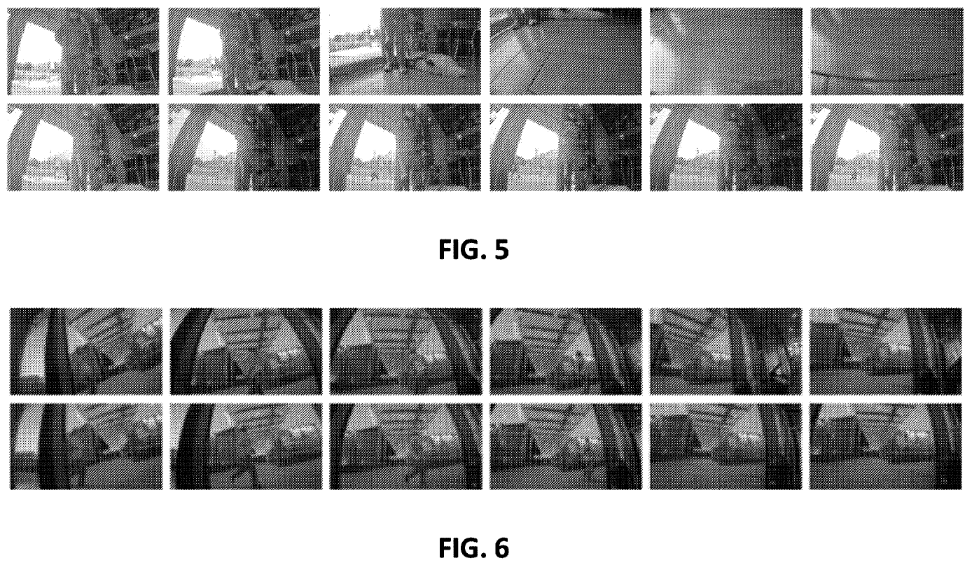

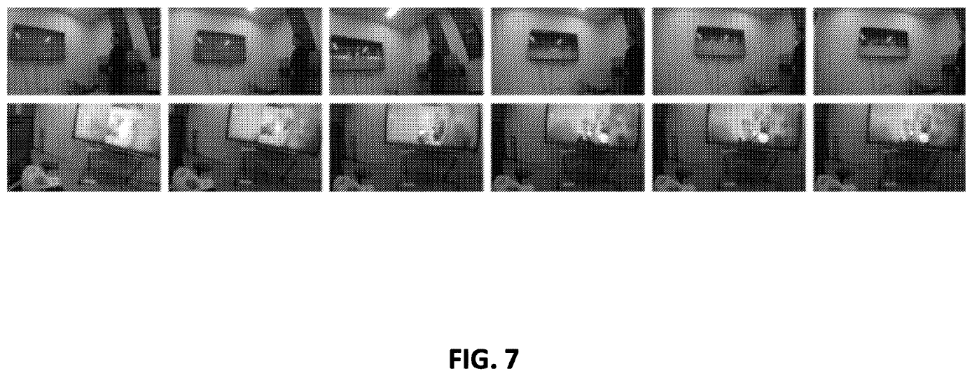

FIGS. 5, 6 and 7 depict three examples of time-series scene image frames for the above three applications—human-dog interaction, dog cognition, and animal-computer interaction, respectively, in the DogsView dataset.

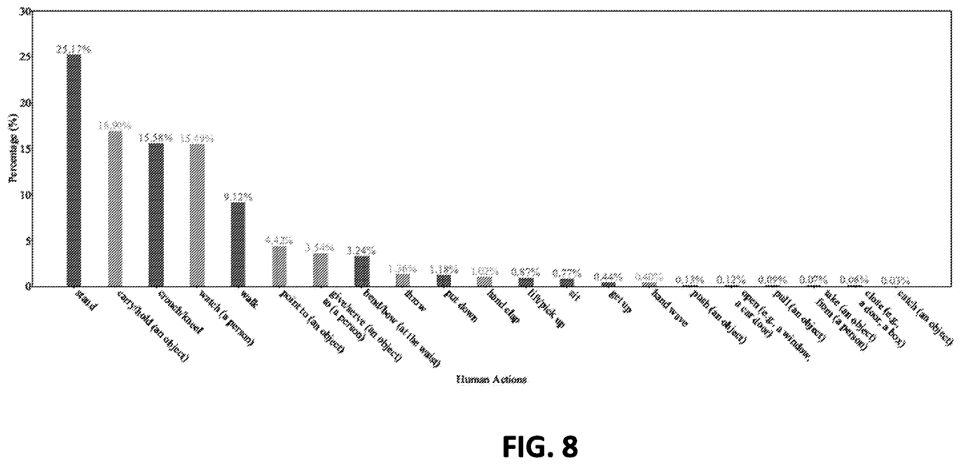

FIG. 8 is a bar chart depicting the ratio of each action type to the number of action signals in the DogsView dataset. In the illustrated example, there are 21 human action categories, including 7 types of human signals commonly used in previous HDI studies and 14 unintentional signals (marked in darker gray shade). The seven types of human signals commonly used in previous HDI studies are: 1) eye-gaze contact or “watch” in this paper, 2) “hand pointing” or “point to” in this paper, 3) “arm extension” (including “carry/hold”, 4) “give/serve (an object) to”, 5) “throw”, 6) hand wave, and 7) hand clap. These seven signals are mostly related to eye/arm/hand movements, and such actions are carefully selected such that the human experimenters can manually identify and analyze them. In aspects, the disclosed system may be configured to recognize human actions, which allows for the analysis of more complicated and more subtle visual signals. Thus, 14 new human actions (more signals may be added in the future) are added that are common in real life but are not well-investigated by previous human-dog interaction works.

The performance of disclosed system has been evaluated on at least three tasks: dog attention recognition, behavior recognition, and causal attention reasoning. Before evaluating the disclosed system, a qualified dataset to train the network is generated. In an aspect, the qualified training dataset comprises: (1) time-aligned videos of the dog's eye region and videos of visual scenes reflecting dogs' field-of-view, and (2) annotations of dogs' visual attention and behaviors. In an example, training data and test data is collected using the disclosed system, and the collected data is (e.g., manually) labeled with appropriate labels. For training and test data collection, a number (e.g., five) dogs (e.g., domestic dogs) may be recruited. In an example, the disclosed smart glasses are put on each dog, and an experimenter is instructed to interact with the dog by showing various communication signals, i.e., body actions, that are intended to communicate with dogs. These signals may include a number (e.g., seven) intentional human behaviors: “watch”, “point to”, “carry/hold”, “give/serve (an object) to”, “throw”, “hand wave”, and “hand clap”. Several involuntary behaviors may also occur during data collection. The seven voluntary signals allow identification of the behavioral causality of dogs with high confidence. In aspects, the disclosed smart eyewear is used to collect data as the experimenter is interacting with each dog. In an example, eye/scene video data with aligned timelines is collected. As just an example, 32 46-minute eye/scene video data with aligned timelines is collected.

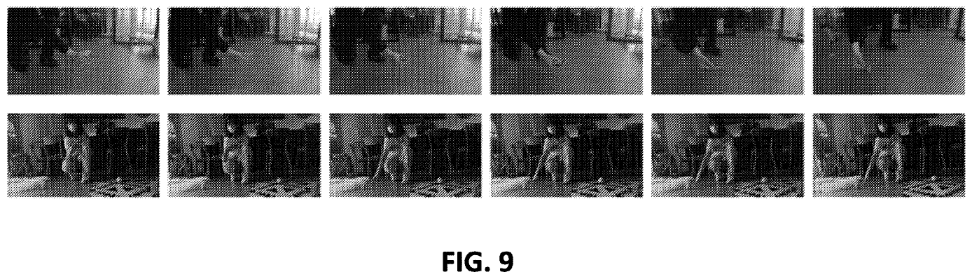

Labeling of the data may involve one or more human annotators manually labeling dogs' frame-level visual attention, following the same criteria of the dog's visual attentive state defined above. The human experimenters may also observe dogs' behaviors during the experiments and record the category of behaviors. The experiments may be carefully such that those causalities can be easily identified with high confidence to obtain the ground truth of behavioral causes. For instance, in a two-choice experiment, if the dog rushes to a food site immediately after the pointing action, it can be reasonably assumed that the action causes its behavior. The experimenters may determine whether the selected cause is correct based on their experiences. In an aspect, to accelerate the labeling process, a labeling tool graphical user interface (GUI), such as a Python GUI, is developed. FIG. 9 depicts two examples of time-series attentive image frames for a dog with different behaviors. In particular, examples of two human communicative signals and the corresponding dogs' different behavioral responses are illustrated. More specifically, the top row of FIG. 9 depicts a scenario in which a person is “carrying/holding (an object)” and a dog is “staring and walking towards”; and the bottom row of FIG. 9 depicts a scenario in which a person is “pointing to (an object)” and a dog is “staring”.

After the disclosed system is trained, the system may be used to examine more scenarios to reveal dogs' visual attention. The data in this stage may be used to construct a dataset, such as the DogsView dataset described above.

Because discovering the casual attention in dogs is an “unseen task”, multiple measures may be needed for performance evaluation. In an example, three aspects of this task may be evaluated: 1). prediction of the dog's attentive state, of which the measures may be accuracy, precision, and recall, 2). prediction of the dog's behavioral responses to visual stimulus, which may be evaluated with multilabel-based measures, such as accuracy, weighted-averaged precision, and weighted-averaged recall, and 3). finding causes of dog behaviors. To quantify the third aspect, in an example, the predicted rationale set, a temporally consistent semantic graph, may be visualized and it may be manually determined whether it has successfully captured the ground truth cause of behavior. In an example, the predicted Rationale Score (RS), an implicit but quantitative indicator of behavioral causality, is also visualized and analyzed as a side measurement of the prediction quality.

Tables 2 and 3 below show the comparison of visual attention recognition performance and dog behavior recognition performance, respectively, of the disclosed system (referred to in tables 2 and 3 as “CANINE”) and several baseline methods. The baseline methods for the visual attention recognition performance include 1) an SG-mask method that jointly uses eye tracking and scene graph method for attention recognition. In contrast to the disclosed system, the SG method is set up for an ablation study without using dog visual masks. 2) An eye-tracking method that is designed to identify dog visual attention using eye tracking only. The semantic meaning of visual content is not used. It first predicts dog gaze points and then identifies the occurrence of visual attention, i.e., when most gaze points are located in a relatively small region for an adequate duration. And 3) saliency method, which uses saliency prediction to estimate dog visual attention. The saliency prediction method estimates the regions that attract viewer attention. As can be seen in table 2, the disclosed system outperforms the baseline methods in terms of Accuracy, Precision, and Recall, indicating the necessity of combining the dog visual mask, dog gaze behavior information, and the semantic meaning of visual stimuli to estimate visual attention.

The baseline methods for dog behavior recognition performance include 1) A “complete” method. Compared with the disclosed system, the complete method is designed to use only the trained complete generator and predictor for dog behavior recognition, in which the complete generator and predictor are disabled. 2) A “complete-mask” method. Like the complete method, the complete-mask method also employs the complete generator and predictor for dog behavior recognition; however, dog visual masks are disenabled. And 3) A “rationale-mask” method. Unlike the disclosed system, the rationale-mask method does not use dog visual masks. As can be seen in table 2, the disclosed system achieves the best performance in terms of Accuracy, Precision, and Recall, demonstrating the effectiveness of using the rationale predictor and dog visual mask.

TABLE 2

| Method | Accuracy | Precision | Recall |
|---|---|---|---|
| CANINE | 80.13% | 72.31% | 70.68% |
| SG-mask | 77.18% | 65.16% | 68.53% |
| Saliency | 56.21% | 40.82% | 53.90% |
| Eye-tracking | 45.86% | 34.82% | 61.56% |

TABLE 3

| Method | Accuracy | Precision1 | Recall² |
|---|---|---|---|
| CANINE | 80.27% | 79.38% | 80.27% |
| Complete | 78.23% | 76.89% | 78.23% |
| Complete-mask | 66.67% | 67.42% | 66.67% |
| Rationale-mask | 67.35% | 67.84% | 67.35% |

Causal attention reasoning of the disclosed system was also evaluated. For example, the disclosed system may be used to discover whether dogs follow humans' misleading signals and why. Previous research found that dogs do not always follow misleading human communicative signals. For example, if one holds an object (e.g., a toy ball) when playing with a dog, the dog will stare at one's hand or the object. If one throws the ball, the dog will likely direct its gaze to where it lands. If one only pretends to throw a ball, will the dog's behavior change? What visual stimuli make dogs follow or ignore human “throwing” actions? This scenario aims to answer the above questions by examining how dogs respond to misleading and straight-forward communicative signals from humans.

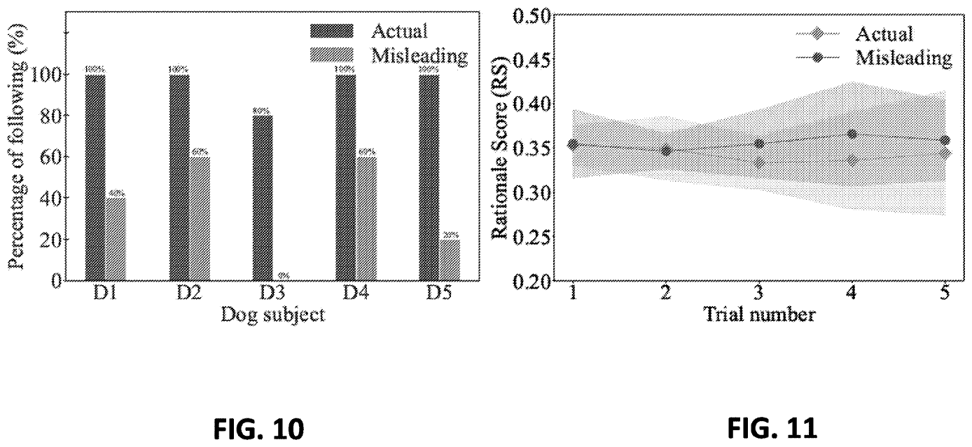

In an experimental procedure, the disclosed smart eyewear may be put on a dog in a room familiar to the dog. A human experimenter may stand in front of the dog, look at the dog, and throw a small bag. This session may include a number (e.g., five) trials for each of a plurality of dogs. The experimenter may then repeat the above process in a number (e.g., five) additional trials. However, during the additional trials, the experimenter may only pretend to throw an object. FIG. 10 illustrates the proportion of five-dog trials in which a dog obeys the two pre-assigned human signals: a straight-forward “throw” behavior and a deceptive “throw”. As can be seen in FIG. 10, all of the five dogs follow the actual “throw” in most trials, while the majority of dogs recognize the experimenter's misleading “throw” and ignore it in most trials.

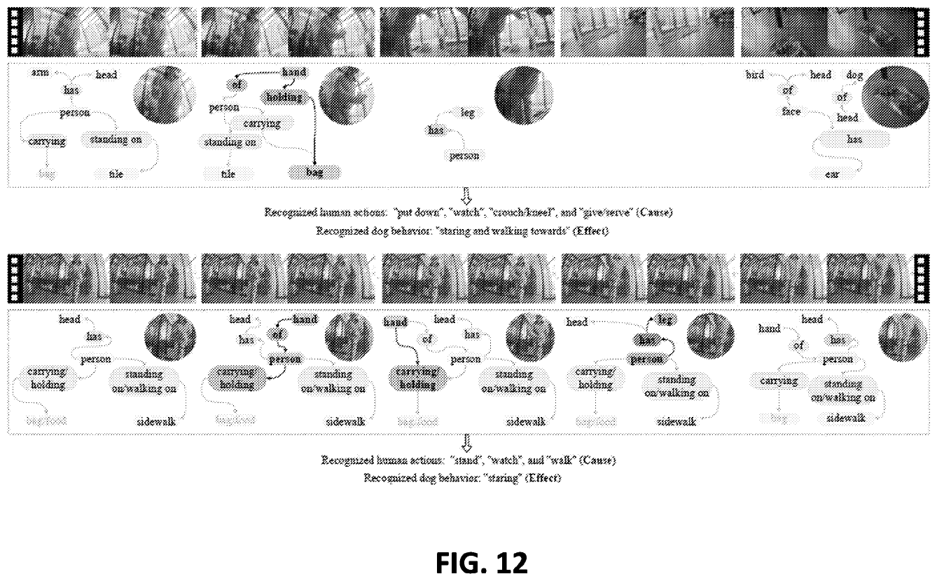

FIG. 11 provides further understanding of how dogs respond to the straight-forward “throw” and the deceptive “throw” by showing the mean Rationale Score (RS). RS reflects the degree of influence visual stimuli have on dog behavior. As can be seen in FIG. 11, the RSs of the deceptive “throw” cases are higher than those of the straight-forward throw cases. Intuitively, dogs can recognize the experimenter's deceitful “throw” action, and therefore, they may stare at the human experimenter to gather further information, resulting in higher RS. The colored background in FIG. 12 indicates the standard deviation of RS in each trial for the five dogs. The variation increases with the number of trials in both the straight-forward “throw” case and the misleading “throw” case. That is in line with our intuition because the dog's physical state differs from trial to trial. In later trials, dogs may become tired. Also, repeated similar communicative signals and interactions with dogs can cause the dog to become fatigued, leading to a big variation in RS.

An analysis of variance (ANOVA) to study the effect of different dogs on the RS response in actual “throw” and misleading “throw” cases, respectively, was also conducted. In each case, five observations for every dog were made. The null hypothesis H₀ for the overall F-test is that all five dogs produce the same RS response, on average. The critical value is F_{4,20}=2.25 at α=0.1000. In the actual “throw” case, F=5.35>2.25 (p=0.0043). The result is significant at the 10% significance level. The null hypothesis was rejected, concluding that there is strong evidence that the expected values in the five dogs differ. In other words, there is a big individual variation of RS among dogs' responses to the straight-forward “throw”. The similar observation can also be found in the misleading “throw” case where F=2.58>2.25 (p=0.0689).

Two illustrative cases from the above ten trials were selected to show the causal attention reasoning of the disclosed system, as shown in FIG. 12. FIG. 12 (top row) shows the following actual action case. From the image frames, it can be seen that the dog first stares at the bag in the experimenter's hand, then the dog walks toward the place where the bag falls. The causal attention inferred by the disclosed system is that the dog is staring and walking towards because of a person's actions: “put down”, “watch”, “crouch/kneel”, and “give/serve”. That cause can also be observed from the semantic graphs that contain the triplets explaining the dog's behavior, e.g., (hand-of-person), (hand-holding-bag). In contrast, FIG. 12 (bottom row) shows a case where the dog does not blindly follow the experimenter's deceptive action. As can be seen, the dog is always staring at the experimenter. The causal attention inferred by the disclosed system is that the dog is staring because of a person's actions: “stand” “watch”, and “walk”. The main triplets that explain the dog's behavior are (hand-of-person), (hand-carrying/holding-), and (person-had-leg).

The disclosed system may be also used to discover How the dogs learn from new/unknown human communicative signals. Dogs are known to have the ability to learn and reason continuously. For example, dogs are adept at interpreting humans' communicative signals, such as hand gestures or gaze contact. They can also learn known signals to rapidly understand new ones. This scenario is designed to examine how dogs perceive known communicative signals and generalize them to new signals. This study compares the known human cue of “hand pointing” with a relatively the under-studied cue of “leg pointing” to understand the dogs' causal attention. More kinds of human cues can be examined in the same way.

In an example experiment procedure, two balls may be placed on each side of an experimenter. The experimenter may stand in front of the dog and looks at the dog. Then, the experimenter may use a finger to points to one of the balls. This process may be repeated a number of (e.g., five) times. The experimenter may then point to a ball with a leg, and the process may also be repeated a number of (e.g., five) times.

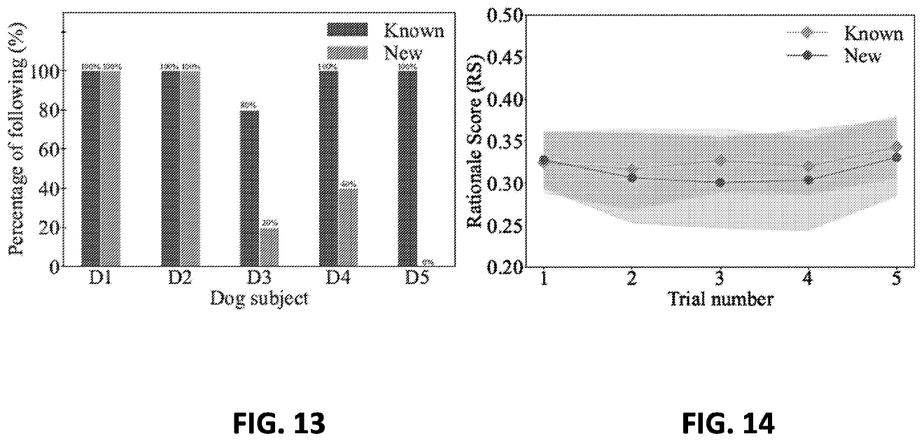

FIG. 13 shows the percentage trials for each of five dogs following two different human signals. One is “hand pointing”, a known signal that dogs are well-known to follow, and the other is “leg pointing”, which is an understudied novel (to the dogs) signal. As can be seen, the five dogs follow “hand pointing” in most trials (80% or higher), while 3 out of 5 dogs do not follow the “leg pointing” in most trials. The reason may be that the three dogs cannot understand the intention cue from “leg pointing” conveyed by human experimenters.

FIG. 14 shows the variation of mean RS in these trials. As can be seen in FIG. 14, the RSS in “hand pointing” cases are consistently higher than those of “leg pointing” cases. Moreover, the variation of RSs for “hand pointing” cases is less than that of “leg pointing” cases. This is because dogs are better at understanding “hand pointing” than “leg pointing”, and therefore, dogs will be more attracted by “hand pointing” for gathering communicative information. Based on the ANOVA analysis, F=4.09>2.25 (p=0.0140) for “hand pointing” case, and F=3.61>2.25 (p=0.0225) for “leg pointing” cases, indicating that there is a large difference in the response degree of the dogs to these two signals.

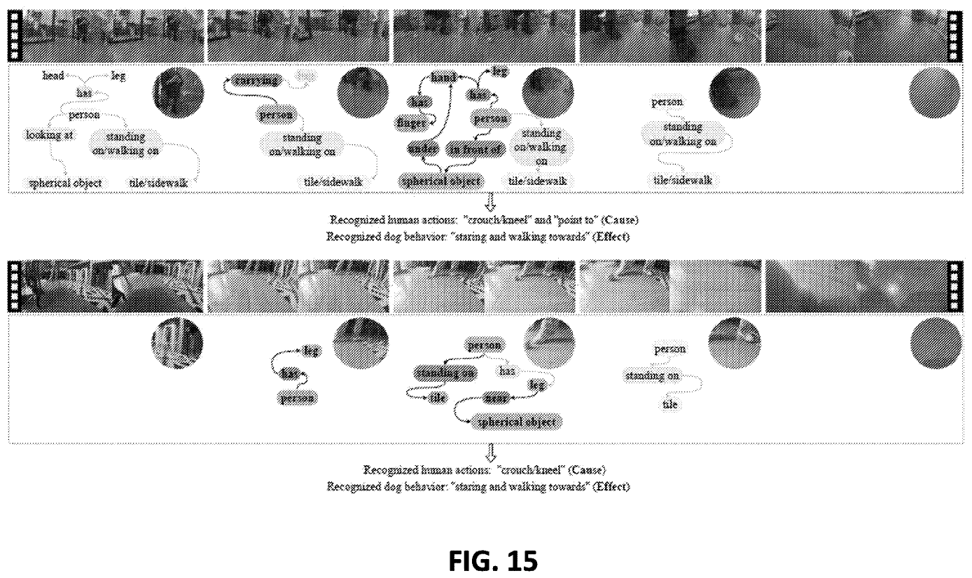

FIG. 15 provides two example trials to study how dogs learn a relatively new human communicative signal “leg pointing” from a well-known one “hand pointing”. FIG. 15 (top row) shows that dog attention focuses on parts of the experimenter's body, e.g., hand, finger, and leg. The causal attention inferred by the disclosed system is that the dog's behavior is “staring and walking towards” because of humans' actions: “crouch/kneel” and “point to”. The semantic graphs provides further insights. For example, in the middle of the attention session, the main triplets reflecting the causes of the dog's behavior are (person-has-hand), (hand-has-finger), (person-in front of-spherical object), and (spherical object-under-hand). FIG. 15 (bottom row) provides an example of a “leg pointing” action. The dog first focuses on the experimenter's leg. Then, the dog notices that the leg is close to a spherical object, and the dog walks toward it. Therefore, CANINE concludes that the dog's behavior is also “staring and walking towards”. However, it states that the human behavior that causes the dog's behavior is “crouch/kneel”.

The disclosed system may be also used to investigate whether dogs make counterproductive choices and why. Previous work has shown that dogs make counterproductive choices when they notice humans' ostensive signals. For example, in a previously conducted food quantity preference test, if humans show an explicit preference for dogs to choose smaller amounts of food, dogs will ignore their nature of choosing larger quantities and follow humans' preference. Using use a food quantity preference task, the disclosed system may to automatically reveal that dogs make counterproductive choices in response to overt cues from humans in an open-world environment.

In an example, in the food quantity preference task two plates of food in a dog's daily room are prepared: one with more food and one with less. The two plates of food may be alternatively shown to the dog and the two plates may then be placed on two sides of an experimenter. An assistant may hold and pet the dog, preventing the dog from eating the food immediately. The experimenter may look at the dog and points to the plate with the smaller amount of food. The assistant may then let the dog go, allowing the dog to choose a plate. This process may be repeated e.g., ten times. The dog may be permitted to rest for a few minutes between trials.

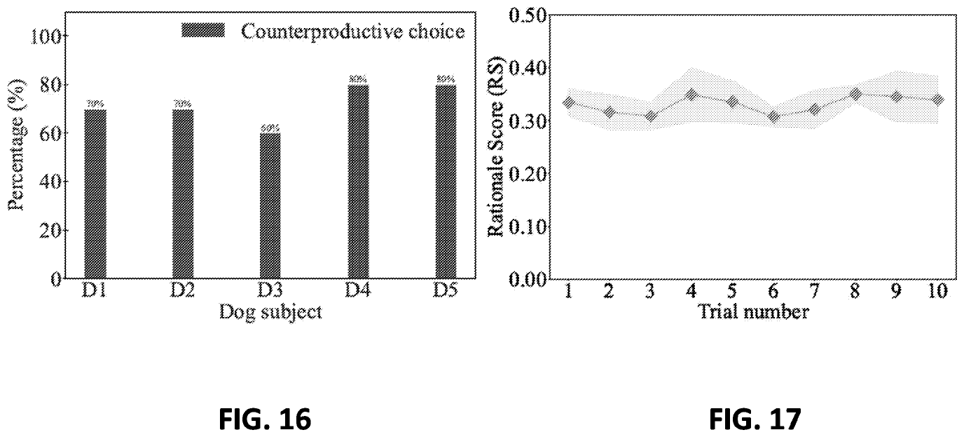

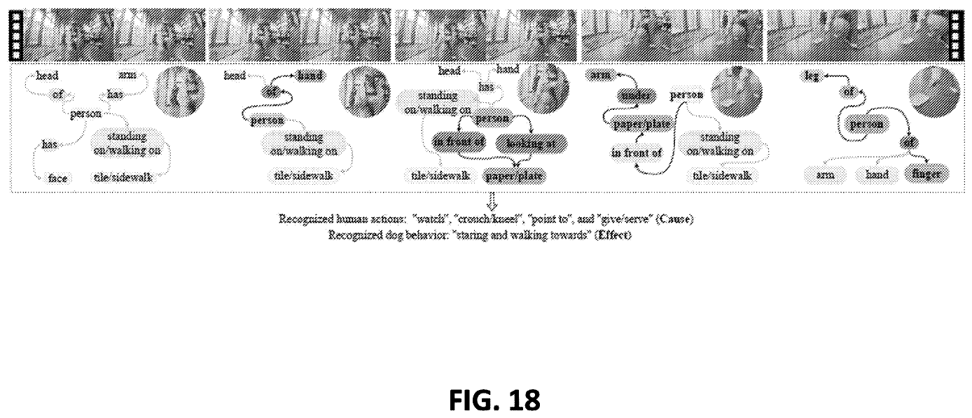

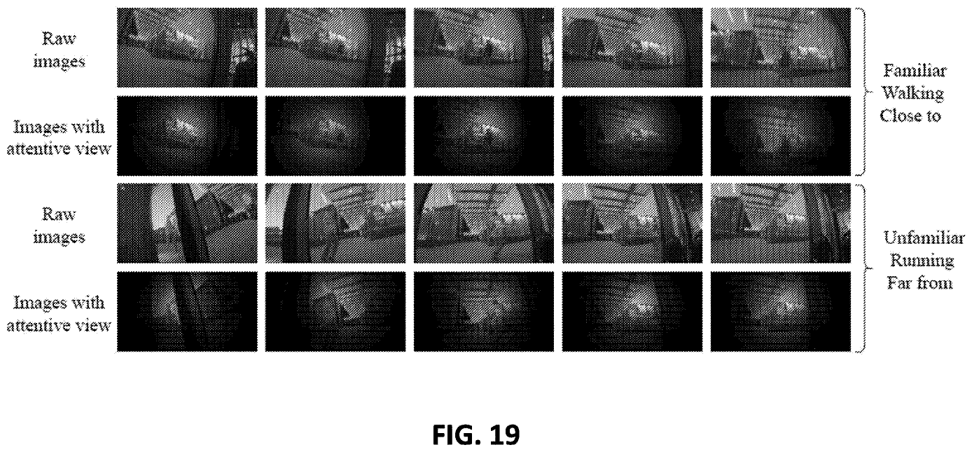

FIG. 16 shows the percent of trials in which every dog makes counterproductive choices in the food quantity preference test. It can be seen that all dogs make counterproductive choices in most trials, which demonstrates that, in most cases, dogs ignore their nature of choosing larger quantities and follow human preference. As can be seen in FIG. 16, mean RS is stable, as the number of trials increases despite fluctuation and has less variation than those in the above two scenarios, which demonstrates that there is a relatively low difference in the response degree of dog subjects (p=0.1004).

FIG. 17 shows an example trial of the food quantity preference test. In this trial, the dog looks and walks toward the smaller amount of food, following the experimenter's explicit body actions, including “watch”, “crouch/kneel”, “point to”, and “give/serve”. As can be seen in FIG. 17, the main time-series triplets that reflect the cause of the dog's behavior in the scene graphs are (person-of-hand), (person-in front of-paper/plate), (person-looking at-paper/plate), (paper/plate-under-arm), and (person-of-finger).

The results of field trials to explore the potential use of the disclosed system in open-world scenarios are now presented. The disclosed system enables the automatic capture of dog visual attention and provides quantitative causal analysis. The disclosed system may be used to support various research topics, including human-dog interaction, dog cognition, dog-computer interaction, and more. Several representative scenarios corresponding to the aforementioned research topics were explored in field trials, including dogs in safeguarding roles, comparing visual attention between dogs and humans, and dogs watching videos.

Next, example procedures and results of three field trials are presented to illustrate how the disclosed system may be used to support various research related to visual cognition in dogs.

The first presented scenario involves dogs in safeguarding roles. Dogs play many roles in our society, and safeguarding has always been one of the most important. Therefore, dog safeguarding is chosen as the first scenario. Domestic dogs naturally assert and protect their private territory. Domestic dogs usually have territorial behavior in their home and yard. They are generally wary of anyone entering their territory and respond differently depending on factors such as whether they are familiar with the intruder. In this context, studying how and why dogs direct their visual cognitive focuses to different objects can help better understand dog visual cognition behind the safeguarding practices.

In an example, the disclosed system is used to capture how and why dogs allocate visual cognitive resources differently to different people passing by or traversing their private territories. To this end, the experiment consists of three steps. (1) The dog is placed indoors, facing a floor-to-ceiling glass window and a glass door to ensure the dog can see people passing by or entering the door. (2) The disclosed system is placed on the dog, and let the dog face the window and the door. (3) A 2 (“familiar person” or “unfamiliar person” with dogs)×6 (“close to the door, running” or “close to the door, walking” or “far away from the door, running” or “far away from the door, walking” or “entering the door, walking” or “entering the door, running”) confounding variable matrix may be summarized and all combinations of these variables may be used to examine the various situations a dog might face.

The disclosed system then infers how dogs visually notice people passing by or entering their private territories by computing RS for different human actions, including “passing by”, “running”, and “entering”. Table 4 shows the results. The key observation is that dogs show the highest RS for familiar participants near the door, and the lowest RS for unfamiliar participants running far from the door.

TABLE 4

| | Running | | Walking | | Entering | |
|---|---|---|---|---|---|---|
| | Far from | Close to | Far from | Close to | Running | Walking |
| Familiar | 0.28 | 0.34 | 0.34 | 0.38 | 0.33 | 0.33 |
| participant | | | | | | |
| Unfamiliar | 0.17 | 0.36 | 0.34 | 0.37 | 0.33 | 0.37 |
| participant | | | | | | |

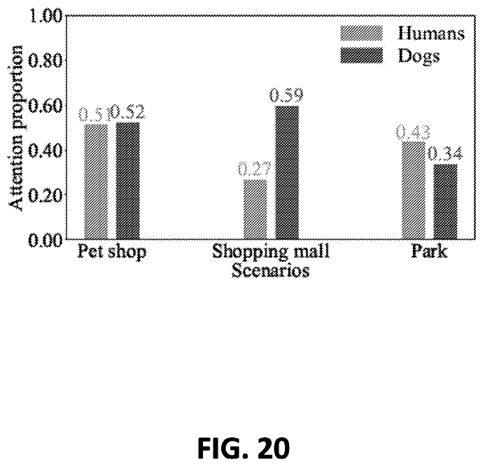

The following case further helps to intuitively explain the reasons behind the observation. Take for example the “familiar participant, close to the door” and “unfamiliar participant, far away from the door”. FIG. 20 shows the time-series of image frames for a dog spotting a familiar passing person who is walking near a door and an unfamiliar person who is running far from the door. As can be seen in FIG. 20, the dog has been staring at the familiar person walking near the door. In contrast, the dog does not stare an unfamiliar person when they walk away. A plausible explanation is that the dog does not recognize the unfamiliar person and therefore does not continue to visually inspect them when they are far away from the door.

The disclosed system can thus help support research on dogs in safeguard roles by providing intuitive and quantitative insights into their reasoning based on casual attention.

The second presented scenario involves comparing visual attention between dogs and humans. Comparing visual cognition between dogs and humans has long been an attractive research topic. As shown in previous work, dogs have similar yet simpler visual cognitive abilities. However, our understanding of their similarities and differences is still very limited. With the disclosed system, our understanding may be broadened and deepened by automatically capturing and quantitatively comparing dog visual attention data in a variety of real-world situations.

Human visual attention may be defined in a similar way to dog visual attention as described above to yield comparable results. Specifically, a human visual attention event may be defined to satisfy the following three conditions: (1) the person's eye movement phase is a fixation or smooth pursuit; (2) there is a fixation target (or informative region) that the person is looking at or a moving target that the person's gaze is steadily following; and (3) the fixation or smooth pursuit phase continues for a period of time. To capture human visual attention. events, smart eyewear for humans may be utilized.

To illustrate how the disclosed system may help explore causal attention in dogs compared to humans, five human participants were recruited into a study to experience a variety of everyday environmental scenarios along with dogs. Each human participant was randomly teamed up with a dog that has participated in previous laboratory experiments. The following three scenarios are selected to explore how the disclosed system may help reveal their causal attention differences: (1) shopping malls, where dogs may be more attracted to the variety of commodities, e.g., persons or stores, than humans; (2) parks, where humans may find something more appealing than dogs; and (3) pet stores, where it is hard to predict how they will be drawn to different things. The dog and human participants are equipped with smart eyewear, each human participant is asked to experience the three environments described while accompanying a leashed dog. In this way, the procedure ensures that both the dog and participant experience a similar visual environment and frequently have overlapping fields of view.

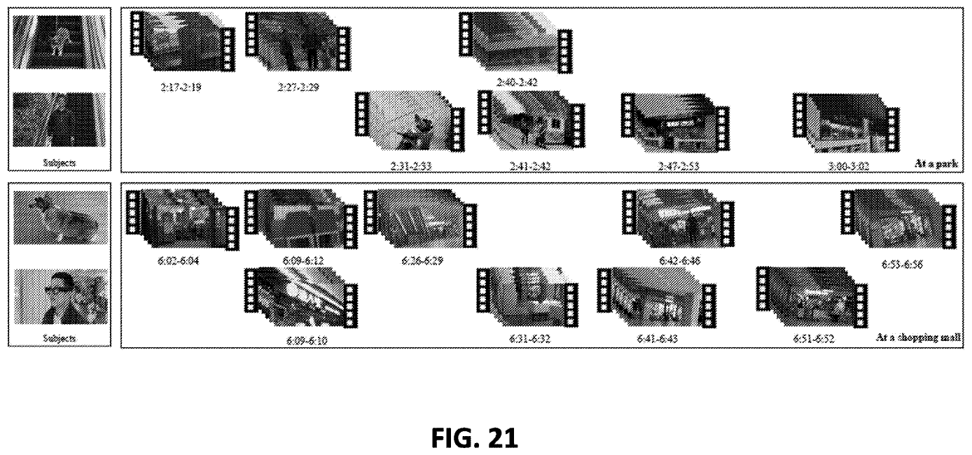

FIG. 21 shows the visual attention proportion of the attentive image frames to the total frames for dogs and humans participating in this study. Dogs have significantly higher attention proportions than humans (0.59 vs. 0.27) at the shopping mall, but the opposite is true at the park (0.34 vs. 0.43). Interestingly, when they are in the pet store, the number of attentive events are similar (0.52 vs. 0.51).

The dogs' attentive content is significantly different, as shown in FIG. 21. In the park, the dog is attracted to persons with more body movements or other inanimate elements, and it ignores the intentional communicative signals from other pedestrians, such as eye contact or waving to the dog. This may indicate that dogs can deliberately ignore human interactive signals and selectively shift attention to their attentive ones.

The third presented scenario involves dogs watching videos. Animal-computer interaction (ACI) is emerging as a subfield of HCI. Dog-video interaction is a popular ACI research topic, e.g., studying dogs' visual habits while watching videos and understanding how dogs interact with video screens. The disclosed system may facilitate investigation and the design of video interactive technologies for dogs, thereby promoting the dogs' welfare.

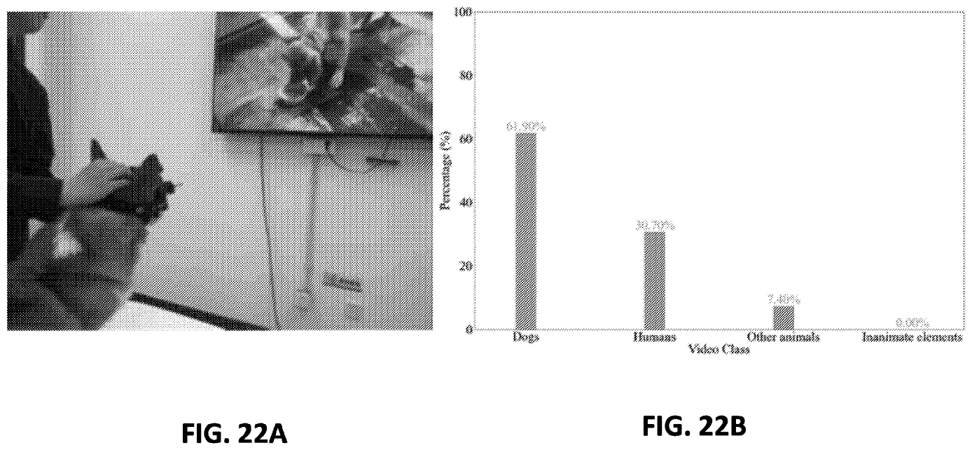

It was explored how the disclosed system can automatically capture and measure visual attention when dogs watch videos. A dog was taken into a room with an LED display playing short videos from the websites such as Tik Tok and YouTube. A person familiar with the dog is in the same room, making sure the dog is comfortable and relaxed. The dog can freely choose whether to watch the video or not. FIG. 22A shows a picture of the dog in this trial.

In an example, a collection of short videos with a total length of 20.42 minutes was played. It was found that the dog only spends 4 minutes watching videos. This result is consistent with expectations and also the findings reported in related studies. For example, it has been shown that human-preferred video content may not attract the attention of dogs. Furthermore, the disclosed system allows us to easily identify and quantify how the different video content attracts dog visual attention and potentially explain why. The collected video content was divided into four categories, socially relevant such as Humans, Dogs, and Other Animals, and non-socially relevant such as Inanimate Elements. FIG. 22B shows the percentage of frames that grab dogs' visual attention as a percentage of the total video frames. As can be seen in FIG. 22B, the video categories that attracts dog attention most frequently are Dogs first, then Humans, followed by Other Animals and Inanimate Elements. Dogs have inherent social characteristics, and while finding their own species more attractive, their attention is also attracted by humans due to their long-term co-living with humans.

The disclosed systems and methods provide a new way to understand cognitive attention in dogs by using smart eyewear to automatically capture the visual attention of dogs in the open world, and further quantify attention events as well as infer related causes. In an example, eye-tracking is the primary step toward successfully uncovering causal attention in dogs. Studies have shown that dogs and humans share many aspects of the visual system. There are also works that directly apply human-based eye-tracking to dogs while using different predetermined thresholds to categorize eye movements for humans and dogs. In aspects of the present disclosure, a human-based eye-tracking method is adopted, and the hyper-parameters are empirically tunes to obtain sufficiently good performance in recognizing dog visual attention. However, affected by many factors such as evolution and environment, dog eye movements differ from those of humans. There are also large differences in eye movement patterns from dog to dog, and these differences are reported to exceed those of humans. Therefore, it is valuable in the future to develop dog-specific eye-tracking approaches capable of enabling more general studies of visual systems and cognition by accommodating individual differences.

In aspects, the definition of dog visual attention is based on assumptions of reference to human visual attention. Furthermore, as discussed above, Gaussian relaxation may be applied to focus the disclosed system on the gaze point to represent areas of potential interest to dogs, and decay to the periphery of dogs' eye view in the form of a circle. The speed and form of this decay may be adjusted, for example based on dog characteristics, such as a breed of the dog, because dogs' visual acuity to the center and periphery region of gaze points may be breed-dependent.

Generally, as shown above, the disclosed eyewear can be easily placed on dogs and used in various scenarios. The dogs may be able successfully perform the predesigned tasks in laboratory experiments. The five recruited dogs as described above make counterproductive choices during the food quantity preference test in more than half of the trials. They are also adept at the signal “hand pointing”, which dogs are generally good at understanding and following. As also shown above, significant individual variation may exist when testing whether the dogs follow misleading signals from humans. For example, dog D3 consistently ignores the deceptive “throw” signal from human experimenters, while D2 and D4 obey the misleading signal most of the time (3 out of 5 trials). Although individual variation is the nature of animals, ubiquitous confounders in the real world, such as the cued objects and spatial location, may bias the study of dog behavioral responses. In some aspects, confounders and summarizing general measurements may be removed for studying dog cognition.

In field studies, the scenario where dogs play safeguarding roles demonstrates how dogs allocate their visual cognitive resources differently to passing humans. It is potentially valuable for studying dog territory defense, territorial aggression, etc. In aspects, the quantitative index, RS, provides an additional perspective. The disclosed system has been used to explore how dogs and humans are attracted by different targets in similar environments, as well as investigate the scenarios where dogs interact with videos. In other aspects, the disclosed system may be used in other scenarios to study dog cognition, e.g., how dogs interact with inanimate elements in non-social settings and how different dog characteristics, such as ages, homes, breeds, and experiences correlate to their visual cognition.

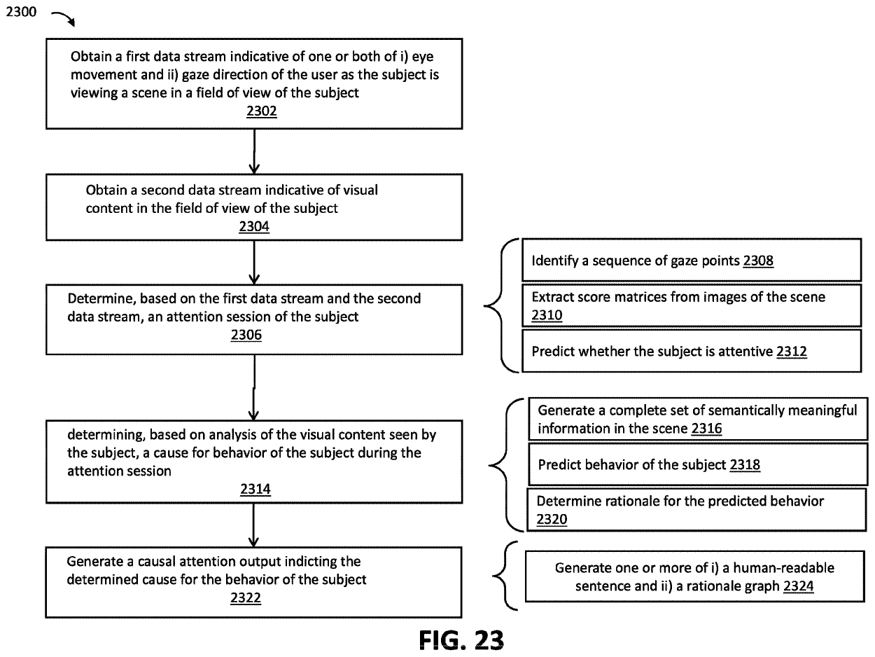

FIG. 23 depicts a method 2300 for determining causal attention of a subject, such as a canine subject, in accordance with one example. The method 2300 may be implemented by one or more of the processors described herein. For instance, the method 2300 may be implemented by an image signal processor implementing an image processing system, such as the system 100 of FIG. 1 or the system 200 of FIG. 2. Additional and/or alternative processors may be used. For instance, one or more acts of the method 2300 may be implemented by an application processor, such as a processor configured to execute a computer vision task.

The method 2300 includes an act 2302 in which one or more procedures may be implemented to obtain a first data stream. The first data stream may be indicative of i) eye movement and ii) gaze direction of the subject as the subject is viewing a scene in a field of view of the subject. The first data stream may include image data that may be obtained from a sensor, such as a camera, for example. The first data stream may include, for example, a plurality of video frames depicting an eye gaze of the subject as the subject is viewing a scene in a field of view of the subject. The first image steam may be obtained from a first sensor, such as an inward-facing camera that may be attached to a smart eyewear frame worn by the subject or other suitable sensor. In an example, the first data stream is obtained from the first sensor 102 of FIG. 1.

At an act 2304, one or more procedures may be implemented to obtain a second data stream. The second data stream may be indicative of visual content in the field of view of the subject. The second data stream may include one or more images or video frames capturing visual content in the field of view of the subject, for example. The second data steam may be obtained from a second sensor, such as a forward-facing camera that may be attached to the smart eyewear frame worn by the subject. In an example, the second data stream may be obtained from the second sensor 104 of FIG. 1.

At an act 2306, the first data stream obtained at the act 2302 and the second data stream obtained at the act 2304 may be processed to detect an attention session of the subject. Detecting the attentions session of the subject at act 2306 may include an act 2308 in which a sequence of gaze points based on the first data stream may be identified. Also, at act 2310, score matrices from images in the second data stream may be extracted. Then, at an act 2312 a prediction on whether the subject is attentive may be made based on the gaze points identified at act 2308 and the score matrices extracted at act 2310. For example, for a time step t, temporal pooling (TP) may be applied to the score matrices extracted at act 2310 to summarize the temporal information in the scene sequence around the time step t. Also, differences between consecutive gaze points identified at act 2308 may be computed to summarize the gaze movement. The temporally reduced score matrix and frame differences of gaze points may be concatenated and then fed into a suitable model, such as a Multi-Layer Perceptron (MLP) model, to produce a binary prediction, i.e., whether the subject is visually attentive in time step t.

At an act 2314, a cause for behavior of the subject may be determined based on analysis of visual content in the scene during the attention session. Determining the cause of behavior of the subject at act 2314 may include an act 2316 at which a complete set of semantically meaningful information may be generated based on the images in the second data stream representing the scene during the attentive session. The complete set generated at act 2316 may comprise a score matrix that includes a plurality of triplets identifying relationships between objects in the scene. Additionally, a rationale set may be generated as a reduced subset of the complete set. The rationale set may be the most compact subset of the complete set that can maximize the predictivity of the subject's behavior, for example.

At an act 2318, the behavior of the subject may be predicted based on the complete set and/or the rationale set. For example, a trained complete predictor may be used to predict behavior of the subject based on the complete set. Additionally, a trained rationale predictor may be used to predict behavior of the subject based on the rationale set. A prediction gap between the behavior of the subject predicted based on the complete set and the behavior of the subject predicted based on the rationale set may be minimized. With the minimized gap, at an act 2320, rationale for the predicted behavior may be revealed.

At an act 2322, a causal attention output may be generated. Generating the causal attention output at act 2322 may include an act 2324 at which one or both of i) a human-readable sentence and ii) a rationale graph may be generated. The human-readable sentence may be composed from the predicted behavior and the revealed rationale for the predicted behavior of the subject. As an example, in an aspect in which the subject is a dog interacting with a human, the human-readable sentence may be in the form of “The dog is performing [a predicted action] because of [the revealed causal attention].” As a more specific example, the human-readable sentence may be “The dog is staring and walking towards because a person is pointing at a paper plate.” The rationale graph, on the other hand, may comprise a graph of a plurality of relationships that may be included in the generated rationale set, for example. The rationale graph may thus depict emergence and changing of the behavior of the subject over time.

In aspects, the disclosed system implementing the method 2300 may be utilized in a variety of applications. For example, the investigation of canine cognitive mechanisms can promote a series of dog-related studies, such as Human-Dog Interaction, dog's social/non-social cognition, and Animal-Computer Interaction. The disclosed system implementing the method 2300 may also provide insight into human cognition and brains, e.g., the theory of how early humans formulate their social awareness can benefit from the study on canine cognition. As another example, the disclosed system implementing the method 2300 may provide insight into human cognitive dysfunctions such as Alzheimer's disease, for example by providing observations of the development of mental deficiencies in dogs. Other academic fields that may benefit from dog cognition studies include the comparative phylogenetics and the ontogenetics. From an application viewpoint, a better grasp of canine cognition may enable improvements in canine training, thus enabling dogs to complete their assigned duties better.

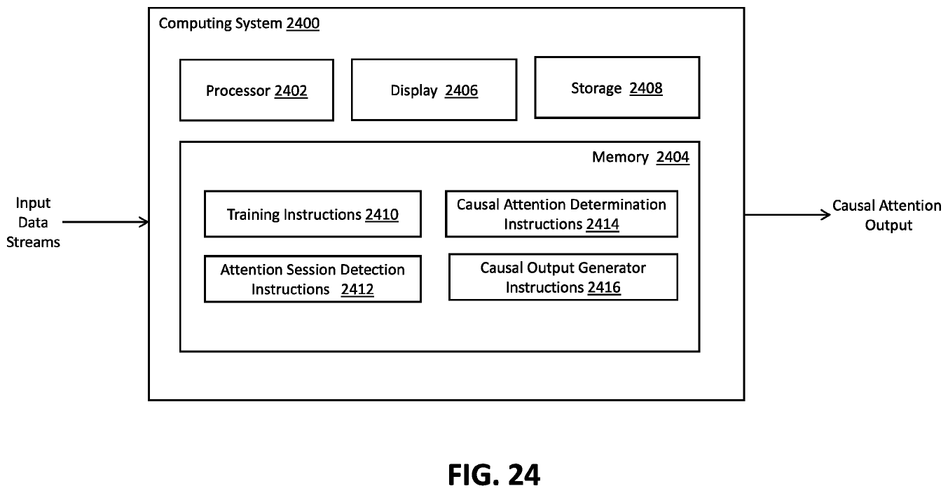

FIG. 24 is a block diagram of a computing system 2400 with which aspects of the disclosure may be practiced. The computing system 2400 includes one or more processors 2402 (sometimes collectively referred to herein as simply “processor 2402”) and one or more memories 2404 (sometimes collectively referred to herein as simply “memory 2404”) coupled to the processor 2402. In some aspects, the computing system 2400 may also include a display 2406 and one or more storage devices 2408 (sometimes collectively referred to herein as simply “storage device 2408” or “memory 2408”). In other aspects, the system 2400 may omit the display 2406 and/or the storage device 2408. In some aspects, the display 2406 and/or the storage device 2408 may be remote from the computing system 2400, and may be communicatively coupled via a suitable network (e.g., comprising one or more wired and/or wireless networks) to the computing system 2400. The memory 2404 is used to store instructions or instruction sets to be executed on the processor 2402. In this example, training instructions 2410, attention session detection instructions 2412, causal attention determination instructions 2414, and causal attention output generator instructions 2416 are stored on the memory 2404. The instructions or instruction sets may be integrated with one another to any desired extent. In an aspect, a set of machine-learned models is stored on the storage device 2408. The set of trained machine models may include a complete predictor, a rationale generator, a rationale generator and/or a causal attention generator as described herein, for example.

The execution of the instructions by the processor 2402 may cause the processor 2402 to implement one or more of the methods described herein. In this example, the processor 2402 is configured to execute the training instructions 2410 to train various models, such as a complete predictor, a rationale generator, a rationale generator and/or a causal attention generator, used for causal attention detection by the computing system 2400. The processor 2402 is configured to execute the attention session detection instructions 2412 to detect an attention session based on data input streams. The processor 2406 is configured to execute the causal attention determination instructions 2414 to determine causal attention in the detected attentions session and generate a causal attention output indicating the causal attention. In various examples, the processor 2406 may be configured to execute the output instructions 2416 to cause the causal attention output to be provided to a user e.g., via the display 2410 and/or to be stored in the storage device 2408, for example.

The computing system 2400 may include fewer, additional, or alternative elements. For instance, the computing system 2400 may include one or more components directed to network or other communications between the computing system 2400 and other input data acquisition or computing components, such as sensors (e.g., an inward-facing camera and a forward-facing camera) that may be coupled to the computing system 2400 and may provide data streams for analysis by the computing system 2400.

The term “about” is used herein in a manner to include deviations from a specified value that would be understood by one of ordinary skill in the art to effectively be the same as the specified value due to, for instance, the absence of appreciable, detectable, or otherwise effective difference in operation, outcome, characteristic, or other aspect of the disclosed methods and devices.

The present disclosure has been described with reference to specific examples that are intended to be illustrative only and not to be limiting of the disclosure. Changes, additions and/or deletions may be made to the examples without departing from the spirit and scope of the disclosure.

The foregoing description is given for clearness of understanding only, and no unnecessary limitations should be understood therefrom.

What is claimed is:

1. A method for determining causal attention of a subject, the method comprising:
obtaining, by a processor, a first data stream indicative of one or both of i) eye movement and ii) gaze direction of the subject as the subject is viewing a scene in a field of view of the subject;
obtaining, by the processor, a second data stream indicative of visual content in the field of view of the subject;
detecting, by the processor, based on the first data stream and the second data stream, an attention session of the subject;
in response to detecting the attention session, determining, by the processor based on analysis of the visual content seen by the subject, a cause for behavior of the subject during the attention session;
generating, by the processor, a causal attention output indicting the determined cause for the behavior of the subject; and
causing, with the processor, the causal attention output to be provided to a user.

2. The method of claim 1, wherein detecting the attention session includes:
identifying, based the first data stream, a sequence of gaze points indicating gaze direction of the subject at a plurality of time steps as the subject is viewing the scene,
extracting, from the second data stream, respective score matrices for respective images in the second data stream at the plurality of time steps, and
predicting, based on the sequence of gaze points and the respective score matrices, whether the subject is attentive at each time step as the subject is viewing the scene.

3. The method of claim 1, wherein predicting whether the subject is attentive at a particular time step comprises:
computing respective differences between consecutive gaze points among the plurality of gaze points,
obtaining temporal information by temporally reducing the respective score matrices corresponding to the gaze points,
generating a binary prediction indicating whether the subject is attentive based on the frame differences of gaze points and the temporal information.

4. The method of claim 1, wherein determining a cause for behavior of the subject during the attention session includes:
generating, based on each image among a plurality of images in the second data stream corresponding to the detected attention session, a complete set comprising a plurality of graph score matrices comprising semantically meaningful information in the scene,
predicting, based on the graph score matrices in the complete set, behavior of the subject viewing the scene, and
determining, based on the graph score matrices in the complete set, a rationale for the predicted behavior of the subject.

5. The method of claim 4, wherein each graph score matrix includes a triplet identifying a first object in the scene, a second object in the scene, and a relationship between the first object and the second object in the scene.

6. The method of claim 4, wherein determining the rationale for the predicted behavior of the subject comprises:
generating a rationale set as subset of the complete set, the subset comprising one or more rationale score matrices that maximize predictability of the behavior of the subject, and
determining the rationale by applying a trained rationale model to the rationale set.

7. The method of claim 4, wherein generating the causal attention output indicting the determined cause for the behavior of the subject comprises generating a human-readable sentence that includes i) the predicted behavior of the subject and ii) the determined rationale for the predicted behavior of the subject.

8. The method of claim 4, wherein generating the causal attention output indicting the determined cause for the behavior of the subject comprises converting the rationale set into a series of temporal scene graphs that illustrate changes in causal attention over time.

9. The method of claim 1, wherein the subject is one of i) a non-human animal subject or ii) a human subject.

10. The method of claim 1, wherein determining a cause for behavior of the subject during the attention session includes determining one or more of i) associations between communicative signals detected in the scene and behavior of the subject, ii) associations between physical stimuli detected in the scene and behavior of the subject and iii) associations between content of videos and desire of the subject to watch the videos.

11. A method for determining causal attention of a subject, the method comprising:
obtaining, by a processor, a first data stream indicative of one or both of i) eye movement and ii) gaze direction of the subject as the subject is viewing a scene in a field of view of the subject;
obtaining, by the processor, a second data stream indicative of visual content in the field of view of the subject;
generating, by the processor, based on the first data stream and the second data stream, a complete set comprising a plurality of graph score matrices comprising semantically meaningful information in the scene;
obtaining, by the processor, a rationale set from the complete set, the rationale set comprising a subset of the graph score matrices that maximizes predictability of the behavior of the subject;
determining, by the processor based on the complete set and the rationale set, causal attention of the subject viewing the scene;
generating, by the processor, a causal attention output indicting the determined causal attention of the subject; and
causing, with the processor, the causal attention output to be provided to a user.

12. The method of claim 11, further comprising, prior to generating the complete set, identifying, by the processor based on the first data stream and the second data stream, ab attention session of the subject viewing the scene, wherein detecting the attention session includes:
identifying, based the first data stream, a sequence of gaze points indicating gaze direction of the subject at a plurality of time steps as the subject is viewing the scene,
extracting, from the second data stream, respective score matrices for respective images in the second data stream at the plurality of time steps, and
predicting, based on the sequence of gaze points and the respective score matrices, whether the subject is attentive at each time step as the subject is viewing the scene.

13. The method of claim 12, wherein predicting whether the subject is attentive at a particular time step comprises:
computing respective differences between consecutive gaze points among the plurality of gaze points,
obtaining temporal information by temporally reducing the respective score matrices corresponding to the gaze point,
generating a binary prediction indicating whether the subject is attentive based on the frame differences of gaze points and the temporal information.

14. The method of claim 11, wherein generating the causal attention output comprises generating a human-readable sentence that includes i) predicted behavior of the subject and ii) determined rationale for the predicted behavior of the subject.

15. The method of claim 11, wherein generating the causal attention output comprises converting the rationale set into a series of temporal scene graphs that illustrate changes in causal attention over time.

16. The method of claim 11, wherein the subject is one of i) a non-human animal subject or ii) a human subject.

17. The method of claim 11, wherein determining a cause for behavior of the subject during the attention session includes determining one or more of i) associations between communicative signals detected in the scene and behavior of the subject, ii) associations between physical stimuli detected in the scene and behavior of the subject and iii) associations between content of videos and desire of the subject to watch the videos.

18. A system comprising:
a first sensor configured to generate data indicative of one or both of i) eye movement and ii) gaze direction of a subject as the subject is viewing a scene in a field of view of the subject;
a second sensor configured to generate data indicative of visual content in the field of view of the subject; and
a processor configured to:
obtain a first data stream indicative of one or both of i) eye movement and ii) gaze direction of the subject as the subject is viewing a scene in a field of view of the subject,
obtain a second data stream indicative of visual content in the field of view of the subject,
detect based on the first data stream and the second data stream, an attention session of the subject;
in response to detecting the attention session, determine, based on analysis of the visual content seen by the subject, a cause for behavior of the subject during the attention session;
generate a causal attention output indicting the determined cause for the behavior of the subject; and
cause the causal attention output to be provided to a user.

19. The system of claim 18, the processor is configured to detect the attention session at least by:
identifying, based the first data stream, a sequence of gaze points indicating gaze direction of the subject at a plurality of time steps as the subject is viewing the scene,
extracting, from the second data stream, respective score matrices for respective images in the second data stream at the plurality of time steps, and
predicting, based on the sequence of gaze points and the respective score matrices, whether the subject is attentive at each time step as the subject is viewing the scene.

20. The system of claim 18, wherein the processor is configured to determine the cause for behavior of the subject during the attention session at least by:
generating, based on each image among a plurality of images in the second data stream corresponding to the detected attention session, a completed set comprising a plurality of graph score matrices comprising semantically meaningful information in the scene,
predicting, based on the graph score matrices in the complete set, behavior of the subject viewing the scene,
generating a rationale set as subset of the complete set, the subset comprising one or more rationale score matrices that maximize predictability of the behavior of the subject, and
determining, based on the graph score matrices in the complete set and the rationale set, a rationale for the predicted behavior of the subject.
